(19) United States
(12) Patent Application Publication
(10) Pub. No.: US 2023/0098686 A1
(43) Pub. Date: Mar. 30, 2023

(54) NERVE SPECIFIC FLUOROPHORE FORMULATIONS FOR DIRECT AND SYSTEMIC ADMINISTRATION

(71) Applicant: OREGON HEALTH & SCIENCE UNIVERSITY, Portland, OR (US)

(72) Inventors: Summer L Gibbs, Portland, OR (US); Lei Wang, Portland, OR (US); Connor W. Barth, Portland, OR (US)

(21) Appl. No.: 17/797,819

(22) PCT Filed: Apr. 9, 2021
(86) PCT No.: PCT/US2021/016663

§ 371 Date: Aug. 5, 2022

Related U.S. Application Data
(60) Provisional application No. 62/971,025, filed on Feb. 6, 2020.

(51) Int. Cl. A61K 49/00

(52) U.S. Cl. CPC A61K 49/0028

(57) ABSTRACT

Nerve-specific fluorophore formulations of Formula (I) for direct or systemic administration are described. The formulations can be used in fluorescence-guided surgery (FGS) to aid in nerve preservation during surgical interventions.

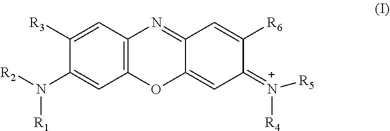

### GOVERNMENT SUPPORT

This invention was made with government support under 1R01EB021362-01A1 awarded by NIH/NIBIB. The government has certain rights in the invention.”

### FIELD OF THE DISCLOSURE

The current disclosure provides nerve-specific fluorophore formulations for direct (local) or systemic administration. The formulations are used in fluorescence-guided surgery (FGS) to aid in nerve preservation during surgical interventions.

### BACKGROUND OF THE DISCLOSURE

Over 300 million surgeries are performed worldwide each year. Despite many recent advances in the treatment of cancer and other diseases, surgery remains the most effective treatment option for a number of diseases and injuries. The ultimate goal of surgery is to remove or repair tissues while minimizing comorbidities by preserving vital structures such as nerves and blood vessels. Recent technological advances including minimally invasive robot assisted laparoscopic surgery have improved outcomes and made it possible to perform difficult procedures robustly with minimal risk. Furthermore, preoperative three-dimensional imaging technologies such as magnetic resonance imaging (MRI) and computed tomography (CT) have vastly improved diagnostic accuracy, staging, and preoperative planning. While advances have been made, identifying vital structures for preservation (e.g., nerves) or tissue for complete resection (e.g., tumors) during surgical procedures remains difficult. Nerve identification and sparing can be difficult intraoperatively due to variations in patient anatomy and often little ability for direct nerve visualization in the surgical field. Currently, intraoperative nerve detection is performed through a combination of naked eye visualization, palpation, and electromyographic monitoring. Several imaging modalities have been utilized in clinical studies for nerve detection including ultrasound, optical coherence tomography, and confocal endomicroscopy. However, these lack specificity, resolution, and wide-field imaging functionality, making it difficult to identify nerve tissues in real time. As a result, nerve damage continues to plague surgical outcomes. Iatrogenic nerve injury affects up to 63 million patients worldwide annually, causing acute and chronic pain as well as impairment or loss of motor and sensory function. Radical prostatectomy (RP), a surgical procedure involving removal of the entire prostate as a prostate cancer cure, is particularly plagued by nerve damage. Furthermore, while minimally invasive methods, such as robotic assisted RP, can achieve equivalent cancer control to open RP while resulting in decreased blood loss, lower transfusion rate, and faster convalescence, these advances provide no benefit in nerve-sparing outcomes and in fact, remove the ability to directly palpate the tissue.

An imaging modality capable of wide field, real time identification of nerve tissues intraoperatively would greatly benefit surgeons in nerve preservation and reduce rates of iatrogenic nerve injury, improving quality of life for patients post-surgery.

In addition to sparing nerves during surgical interventions, the ability to detect the edge of tumors is also incredibly important in clinical medicine. For example, post-surgical tumor margin status is one of the most important prognostic factors for local cancer recurrence and is considered the main measure of a tumor resection's success. Patients undergoing breast conserving surgery (BCS), the most common treatment option for patients with early stage breast cancer, are left with involved or close surgical margins 20-60% of the time, determined by pathological assessment following completion of the surgery. Involved or close margins require follow up re-excision surgery and result in negative patient outcomes.

The current intraoperative guidance techniques, or lack thereof, handicap surgeons' ability to successfully complete the goals of a procedure. Therefore, an imaging modality that can provide intraoperative guidance by highlighting important structures (e.g., nerves) or tissues (e.g., tumor) would greatly benefit surgical outcomes and significantly reduce comorbidities.

Fluorescence-guided surgery (FGS) has the potential to revolutionize surgery by enhancing visualization of specific tissues intraoperatively, effectively bridging the gap between preoperative imaging and surgical guidance. Using optical imaging of targeted fluorescent probes, FGS offers sensitive, real-time, wide-field imaging using compact imaging systems that are easily integrated into the operating room. Several imaging systems have already been developed for FGS applications and are in use clinically. See, for instance: Lee et al. Plastic and reconstructive surgery 126, 1472-1481 (2010); Tummers et al. European journal of surgical oncology: the journal of the European Society of Surgical Oncology and the British Association of Surgical Oncology 40, 850-858 (2014); Troyan et al. Annals of surgical oncology 16, 2943-2952 (2009); Ashitate et al. The Journal of surgical research 176, 7-13 (2012); Verbeek et al. The Journal of urology 190, 574-579 (2013); Gibbs-Strauss et al. Molecular imaging 10, 91-101 (2011); Hirche et al. Surgical innovation 20, 516-523 (2013); Gotoh et al. Journal of surgical oncology 100, 75-79 (2009); and Kitagawa et al. Anticancer research 35, 6201-6205 (2015). Utilizing near-infrared (NIR) light (650-900 nm), many of these systems can identify targeted tissue at millimeter to centimeter imaging depths due to the increased tissue penetration of light and minimal background autofluorescence at these wavelengths (FIG. 1). Additionally, the use of NIR light allows these systems to be implemented in the surgical field without affecting conventional white light visualization. FGS have been successfully utilized in a wide variety of clinical applications to improve surgical outcomes, including tumor resection, sentinel lymph node mapping, angiography, lymphography, and ureter and bile duct anatomic imaging. See, for instance: Kitai et al. Breast Cancer 12, 211-215 (2005); Peek et al. Future Oncol 13, 455-467 (2017); Jeschke et al. Urology 80, 1080-1086 (2012); Chang et al. Plastic and reconstructive surgery 132, 1305-1314 (2013); Yamamoto et al. Eur J Vasc Endovasc Surg 43, 426-432 (2012); Boni et al. Surg Endosc 29, 2046-2055 (2015); Stummer et al. Neurosurgery 42, 518-525; discussion 525-516 (1998); Stummer et al. Lancet Oncol 7, 392-401 (2006); van Dam et al. Nat Med 17, 1315-1319 (2011); van der Vorst et al. World J Gastrointest Surg 4, 180-184 (2012); Al-Taher et al. J Laparoendosc Adv Surg Tech A 26, 870-875 (2016); Ankersmit et al. Surgical innovation 24, 245-252 (2017); and Samkoe et al. Cancer Control 25, 1073274817752332 (2018). For example, FGS applications in glioma resection using 5-aminolevulinic acid (5-ALA) and its fluorescent metabolite protoporphyrin IX (PpIX) has significantly enhanced complete resection rates and revolutionized neurosurgical treatment of brain tumors over the past decade (Hadjipanayis et al. Neurosurgery 77, 663-673 (2015)).

While the promise of FGS has been demonstrated in a variety of clinical and preclinical applications over the past several decades, few efforts in clinical translation of new targeted imaging agents for FGS have been successful, largely due to the enormous regulatory challenge and cost of introducing diagnostic imaging agents into the clinic. Only four fluorescent contrast agents are clinically approved, three of which were grandfathered in from their application as colorimetric dyes prior to the widespread use of fluorescence imaging (Gibbs. Quantitative imaging in medicine and surgery 2, 177-187 (2012)). Of these four dyes, only two, indocyanine green (ICG) and methylene blue, emit NIR fluorescence and both are blood pool agents. There is thus a great need for the development and clinical translation of tissue-specific fluorescent contrast agents for FGS.

Systemically administered probes are subject to the body's biodistribution and clearance, which can mask binding due to low levels of accumulation of a microdose in the targeted tissue. One method for ensuring adequate binding in the tissue of interest and improving sensitivity is local or direct administration at the surgical site. By directly applying the fluorescent probe to the tissues of interest within the surgical field, selective labeling can be attained with a significantly lower dose than systemic administration, yielding equivalent to higher intensity fluorescence signal in the tissue of interest. Aside from significantly lowering the required dose for signal, direct (local) administration provides rapid and highly selective staining, which is beneficial for certain applications to avoid unwanted background that can be caused upon systemic administration.

FGS is well suited to aid in the preservation of vital nerve structures. Direct administration is an especially suited administration strategy for fluorescence-guided nerve sparing RP because nerve labeling via systemic administration would generate high background from nerves in the prostate, which are not able to be spared, and renal nerve-specific fluorophore clearance generates significant fluorescence in the urine within the adjacent bladder (Barth and Summer. Theranostics (2016); Tewari et al. BJU international 98, 314-323 (2006); Patel et al. Eur Urol 61, 571-576 (2012); Barth & Gibbs. Theranostics 7, 573-593 (2017)).

Currently, no NIR nerve-specific fluorophore exists and further fluorophore development is required to obtain a proper candidate for clinical translation. Several classes of nerve specific fluorophores have been studied for FGS. See, for instance: Gibbs-Strauss et al. Molecular imaging 10, 91-101 (2011); Wu et al. Journal of medicinal chemistry 51, 6682-6688 (2008); Wang et al. The journal of histochemistry and cytochemistry: official journal of the Histochemistry Society 58, 611-621(2010); Gibbs et al. PloS one 8, e73493 (2013); Stankoff et al. Proceedings of the National Academy of Sciences of the United States of America 103, 9304-9309 (2006); Cotero et al. Molecular imaging and biology: MIB: the official publication of the Academy of Molecular Imaging 14, 708-717 (2012); Cotero et al. PloS one 10, e0130276 (2015); Bajaj et al. The journal of histochemistry and cytochemistry: official journal of the Histochemistry Society 61, 19-30 (2013); Gibbs-Strauss et al. Molecular imaging 9, 128-140 (2010); Meyers et al. The Journal of neuroscience: the official journal of the Society for Neuroscience 23, 4054-4065 (2003); Wang et al. The Journal of Neuroscience: the official journal of the Society for Neuroscience 31, 2382-2390 (2011); and Park et al. Theranostics 4, 823-833 (2014). Of these, Oxazine 4 is the most promising candidate for development, showing high nerve-specificity and red shifted absorption and emission spectra close to the NIR (Park et al. Theranostics 4, 823-833 (2014)).

FGS offers a rapid and accurate approach to intra-surgical margin assessment that does not compromise tissue integrity. Direct administration of tumor-specific fluorescent probes to resected specimens is an attractive alternative to systemic administration that incurs no risks of toxicity to the patient and would provide an extremely rapid route to clinical use. However, early efforts to stain tissues in this manner resulted in high amounts of non-specific uptake and poor tumor to normal tissue contrast. However, using a dual probe staining approach, containing a targeted and untargeted probe, non-specific uptake can be corrected and highly specific tumor signal achieved (Davis et al. Opt Lett 38, 5184-5187 (2013); Barth et al. Theranostics 7, 4722-4734 (2017)).

Additionally, direct administration methods can be utilized in retrospective analysis of excised tissues such as tumors for tumor margin detection, allowing for indirect, yet still rapid FGS and post-surgical diagnostics.

### SUMMARY OF THE DISCLOSURE

The current disclosure describes work undertaken to develop novel fluorescent probe formulations and local administration/direct application to a tissue of interest and systemic administration methods for FGS not subject to the drawbacks noted in the Background. This disclosure provides significant progress in the development and characterization of clinically viable administration methods and novel imaging probes for nerve identification and tumor margin detection.

Particular embodiments include a gel-based formulation including a nerve-specific fluorophore to facilitate improved signal to background ratio (SBR) for direct administration during surgery through reduction in background staining. In particular embodiments, the gel-based formulations are liquid at room temperature but become a viscous gel upon contact with body temperature. For nerve staining at a surgical site, this characteristic is useful because it diminishes the spread of the applied fluorophore, improving overall SBR through background reduction.

For the gel-based formulation for direct administration, all excipients are FDA approved for human use and the formulation increases the solubility of nerve-specific fluorophores (e.g., LGW01-08) to clinically relevant concentrations. These formulations can be applied directly to the tissue of interest, where they undergo Sol-Gel transition at the site of application. This property is important for the formulation syringe-ability and retention at the tissue of interest. In particular embodiments, the sol-gel transition occurs within, e.g., 1-2 minutes (or, less than 30 seconds) after application, which is important to its practical use in a surgical setting. Further, the formulation can be removed from the tissue easily by washing the tissue with a pharmaceutically acceptable irrigation solution such as saline. These formulations can be scaled-up and produced under GMP conditions.

Also disclosed are two additional clinically-relevant formulations including a nerve-specific fluorophore for systemic administration. One formulation for systemic administration includes cyclodextrin (the main excipient is (2-hydroxypropyl)-β-cyclodextrin) and the second formulation for systemic administration includes a DSPE-PEG micelle. The main excipient in this formulation is N-(methylpolyoxyethylene oxycarbonyl)-1,2-distearoyl-sn-glycero-3-phosphoethanolamine, sodium salt.

In formulations for systemic administration, all excipients are FDA approved for human use, and both formulations increase the solubility of nerve-specific fluorophores to a clinically relevant concentration. Both of the formulations provide clinically relevant pharmacokinetic profiles and clinically acceptable safety (acute toxicity study) profiles for nerve-specific fluorophores. The formulations developed for systemic administration produce the required clinical effect, specifically nerve-specific fluorophore accumulation in the nerve of interest at the desirable SBR. Furthermore, both formulations can be scaled-up and produced under GMP conditions.

### BRIEF DESCRIPTION OF THE DRAWINGS

### Detailed Description

Nerve damage plagues surgical outcomes, significantly affecting post-surgical quality of life. Despite the practice of nerve sparing techniques for decades, intraoperative nerve identification and sparing remains difficult and success rates are strongly correlated with surgeon experience level and ability to master the technique (Walsh & Donker. The Journal of urology 128, 492-497 (1982); Ficarra et al. Eur Urol 62, 405-417 (2012); Damber & Khatami. Acta oncologica 44, 599-604 (2005)). Fluorescence-guided surgery (FGS) shows promise for enhanced visualization of specifically highlighted tissue, such as nerves and tumor tissue, intraoperatively. FGS using optical imaging technology is capable of real-time, wide field identification of targeted tissues with high sensitivity and specificity from tissue targeted fluorescent probes. See, for instance: Frangioni. Journal of clinical oncology: official journal of the American Society of Clinical Oncology 26, 4012-4021 (2008); Gibbs. Quantitative imaging in medicine and surgery 2, 177-187 (2012); Gioux et al. Molecular imaging 9, 237-255 (2010); Vahrmeijer et al. Nature reviews. Clinical oncology 10, 507-518 (2013); and Nguyen et al. Nature reviews. Cancer 13, 653-662 (2013). Operating in the near-infrared (NIR) optical window (650-900 nm wavelengths) where tissue chromophore absorbance, autofluorescence and scattering are minimal, FGS technologies have the ability to identify targeted tissues at millimeter to centimeter depths against a black background (Chance. Annals of the New York Academy of Sciences 838, 29-45 (1998); Gibbs. Quantitative imaging in medicine and surgery 2, 177-187 (2012)).

Several imaging systems have been developed for FGS applications. see, for instance: Lee et al. Plastic and reconstructive surgery 126, 1472-1481 (2010); Tummers et al. European journal of surgical oncology: the journal of the European Society of Surgical Oncology and the British Association of Surgical Oncology 40, 850-858 (2014); Troyan et al. Annals of surgical oncology 16, 2943-2952 (2009); Ashitate et al. Real-time simultaneous near-infrared fluorescence imaging of bile duct and arterial anatomy. The Journal of surgical research 176, 7-13 (2012); Verbeek et al. The Journal of urology 190, 574-579 (2013); Gibbs-Strauss et al. Molecular imaging 10, 91-101 (2011); Hirche et al. Surgical innovation 20, 516-523 (2013); Gotoh et al. Journal of surgical oncology 100, 75-79 (2009); and Kitagawa et al. Anticancer research 35, 6201-6205 (2015); Importantly, the da Vinci surgical robot, frequently used for robotic assisted radical prostatectomy (RP), can be equipped with an FDA approved fluorescence imaging channel.

Direct administration (also sometimes referred to as local administration) is an attractive alternative to systemic administration of fluorescent probes for minimizing potential toxicity and easing regulatory burdens for first in human clinical studies. By selectively labeling tissues within the surgical field, direct administration requires a significantly lower dose than systemic administration. A direct administration methodology has been developed that provides equivalent nerve signal to background (SBR) to systemic administration following a 15-minute staining protocol. Barth & Gibbs. Theranostics 7, 573-593 (2017). This methodology has been successfully applied to autonomic nerve models, which closely mimic the nerves surrounding the prostate. This method has additional benefits in the application to RP since nerve labeling via systemic administration during RP would generate high background from nerves in the prostate, which are not able to be spared, and renal fluorophore clearance, producing significant fluorescence signal in the urine within the adjacent bladder. Both of these extraneous fluorescence signals would diminish the ability to identify the cavernous nerves within the neurovascular bundle (NVB), which are responsible for continence and potency (Barth and Summer. Theranostics (2016). Tewari et al. BJU international 98, 314-323 (2006); Patel et al. Eur Urol 61, 571-576 (2012)). Perhaps most importantly, the direct administration methodology requires 16 times lower dose than systemic administration and when scaled to humans by body surface area the dose falls within the requirements for clinical translation under an exploratory investigational new drug (eIND) application to the FDA. Studies conducted under an eIND require minimal preclinical toxicity testing, since only a microdose (<100 μg) is administered to each patient, significantly reducing the cost of first-in-human studies.

While the direct administration methodology has provided high nerve specificity and SBR with a short staining protocol in preclinical rodent models (Barth & Gibbs. Theranostics 7, 573-593 (2017)), preliminary staining studies in large animal models generated significant background. To facilitate clinical translation, an improved formulation strategy that is FDA approved and facilitates increased application control for staining a variety of tissue surfaces, angles, and morphologies will be required.

Several classes of nerve specific fluorescence imaging probes have been studied preclinically for FGS. See, for instance: Gibbs-Strauss et al. Molecular imaging 10, 91-101 (2011); Wu et al. Journal of medicinal chemistry 51, 6682-6688 (2008); Wang et al. The journal of histochemistry and cytochemistry: official journal of the Histochemistry Society 58, 611-621 (2010); Gibbs et al. PloS one 8, e73493 (2013); Stankoff et al. Proceedings of the National Academy of Sciences of the United States of America 103, 9304-9309 (2006); Cotero et al. Molecular imaging and biology: MIB: the official publication of the Academy of Molecular Imaging 14, 708-717 (2012); Cotero et al. PloS one 10, e0130276 (2015); Bajaj et al. The journal of histochemistry and cytochemistry: official journal of the Histochemistry Society 61, 19-30 (2013); Gibbs-Strauss et al. Molecular imaging 9, 128-140 (2010); Meyers et al. The Journal of neuroscience: the official journal of the Society for Neuroscience 23, 4054-4065 (2003); Wang et al. The Journal of neuroscience: the official journal of the Society for Neuroscience 31, 2382-2390 (2011); Park et al. Theranostics 4, 823-833 (2014). Of these, oxazine fluorophores (e.g., Oxazine 4) have demonstrated the most promise for clinical translation, with high nerve specificity following both direct and systemic administration. LGW01-08 is a particularly promising compound and was chosen as the lead compound for advancement to clinical studies. Although LGW01-08 has been shown to demonstrate high nerve specificity and adequate fluorescence signal for real time imaging, previous studies have been conducted utilizing a co-solvent formulation as a vehicle for intravenous injection (Gibbs-Strauss et al. Molecular imaging 10, 91-101 (2011); Barth & Gibbs. Theranostics 7, 573-593 (2017)). The co-solvent formulation is only stable at room temperature for <30 minutes, cannot solubilize concentrations above 5 mg/mL, and requires the use of dimethyl sulfoxide and Kolliphor EL as solubilizing agents, which hampers clinical translation due to vehicle induced toxicity issues. Additionally, the co-solvent formulation is liquid based and thus not ideal for staining angled or vertical tissue surfaces. Therefore, a clinically viable formulation with FDA approval was needed for direct administration and intravenous injection of nerve-specific fluorescence for FGS.

The current disclosure describes clinically relevant formulation strategies for direct administration and intravenous injection of relevant compounds, such as the fluorophores described herein.

In particular embodiments, the fluorophores are compounds of Formula (I):

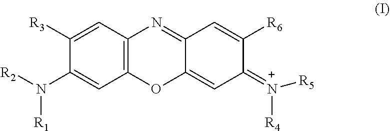

wherein:

R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:

a)

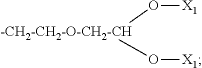

b)

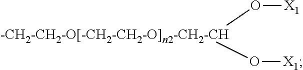

c)

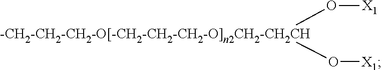

and
d)

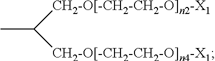

R₃ is hydrogen or R₂ and R₃ together form a fused ring, creating a core of Formula (II):

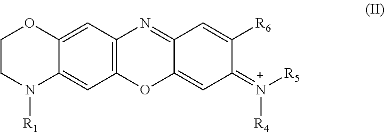

R₄ and R₅, together with the nitrogen atom to which they are bound, form a ring selected from the group of:

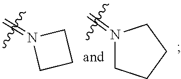

or, when the compound is of Formula (II), R₄ and R₅ may be independently selected from C₁-C₆ alkyl, with the proviso that, when R₁ is methyl and R₄ is ethyl, R₅ is not ethyl;

R₆ is hydrogen;

or, when R₂ and R₃ together form a fused ring to create a core of Formula (II), R₅ and R₆ may also, together with the nitrogen atom to which R₅ is bound, form a fused ring, creating a core of Formula (III):

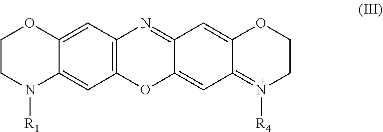

with the proviso that, when R₄ and R₅, together with the nitrogen atom to which they are bound, for the ring

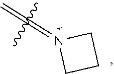

and R₃ is H, R₁ and R₂, together with the nitrogen atom to which they are bound, may form a pyrrolidinyl ring;

X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;

n is an integer selected from the group of 1 and 2;

n1 is an integer independently selected in each instance from the group of 1, 2, 3, and 4; n2 is an integer independently selected in each instance from the group of 1, 2, 3, 4, 5, 6, 7, 8, 9, and 10;

n3 is an integer independently selected in each instance from the group of 1, 2, 3, 4, 5, 6, 7, 8, 9, and 10;

n4 is an integer independently selected in each instance from the group of 1, 2, 3, 4, 5, 6, 7, 8, 9, and 10; and

with the proviso that the sum of n2+n2 is not greater than 10;

with the proviso that the sum of n2+n3 is not greater than 10;

with the proviso that the sum of n2+n4 is not greater than 10;

with the proviso that the sum of n3+n4 is not greater than 10;

with the proviso that, when R₄ and R₅, together with the nitrogen atom to which they are bound, form the ring

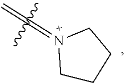

R₁ and R₂ are not both methyl, R₁ and R₂ are not both ethyl, R₁ and R₂ are not both n-propyl, R₁ and R₂ are not both n-butyl, and R₁ and R₂ are not both n-pentyl; and

with the proviso that, when the compound is of Formula (III), when R₁ is methyl, R₄ is not methyl.

A second embodiment provides a compound of Formula (IV):

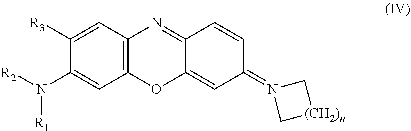

wherein:

R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:

a)

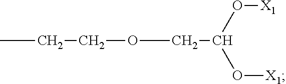

b)

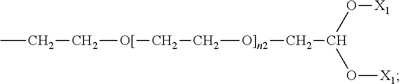

c)

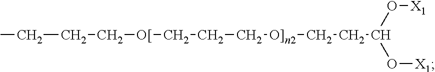

and
d)

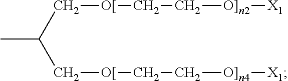

R₃ is hydrogen or R₂ and R₃ together form a fused ring, creating a core of Formula (V):

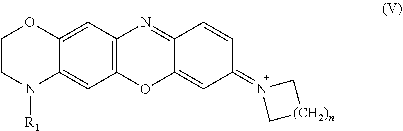

with the proviso that, when n is 1, and R₃ is H, R₁ and R₂, together with the nitrogen atom to which they are bound, may form a pyrrolidinyl ring;

X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;

n is an integer selected from the group of 1 and 2;

n1 is an integer independently selected in each instance from the group of 1, 2, 3, and 4;

n2 is an integer independently selected in each instance from the group of 1, 2, 3, 4, 5, 6, 7, 8, 9, and 10;

n3 is an integer independently selected in each instance from the group of 1, 2, 3, 4, 5, 6, 7, 8, 9, and 10;

n4 is an integer independently selected in each instance from the group of 1, 2, 3, 4, 5, 6, 7, 8, 9, and 10; and

with the proviso that the sum of n2+n2 is not greater than 10;

with the proviso that the sum of n2+n3 is not greater than 10;

with the proviso that the sum of n2+n4 is not greater than 10;

with the proviso that the sum of n3+n4 is not greater than 10; and

with the proviso that, when n is 2, R₁ and R₂ are not both methyl, R₁ and R₂ are not both ethyl, R₁ and R₂ are not both n-propyl, R₁ and R₂ are not both n-butyl, and R₁ and R₂ are not both n-pentyl.

Within each of the specific embodiments herein, there is a further embodiment that provides a compound as defined by all variables and provisos for the specific embodiment in question, with the further proviso that none of the sum of n1+n2, the sum of n1+n3, and the sum of n1+n4 are greater than 10.

Within each of the specific embodiments herein, there is a further embodiment that provides a compound as defined by all variables and provisos for the specific embodiment in question, with the further proviso that none of the sum of n1+n2, the sum of n1+n3, and the sum of n1+n4 are greater than 8.

Within each of the specific embodiments herein, there is a further embodiment that provides a compound as defined by all variables and provisos for the specific embodiment in question, with the further proviso that none of the sum of n1+n2, the sum of n1+n3, and the sum of n1+n4 are greater than 6.

Within each of the specific embodiments herein, there is a further embodiment that provides a compound as defined by all variables and provisos for the specific embodiment in question, with the further proviso that none of the sum of n1+n2, the sum of n1+n3, and the sum of n1+n4 are greater than 4.

Within each of the specific embodiments herein, there is a further embodiment that provides a compound as defined by all variables and provisos for the specific embodiment in question, with the further proviso that none of the sums in the group of the sum of n1+n2, the sum of n1+n3, and the sum of n1+n4 are greater than 10.

Within each of the specific embodiments herein, there is a further embodiment that provides a compound as defined by all variables and provisos for the specific embodiment in question, with the further proviso that none of the sums in the group of the sum of n1+n2, the sum of n1+n3, and the sum of n1+n4 are greater than 8.

Within each of the specific embodiments herein, there is a further embodiment that provides a compound as defined by all variables and provisos for the specific embodiment in question, with the further proviso that none of the sums in the group of the sum of n1+n2, the sum of n1+n3, and the sum of n1+n4 are greater than 6.

Within each of the specific embodiments herein, there is a further embodiment that provides a compound as defined by all variables and provisos for the specific embodiment in question, with the further proviso that none of the sums in the group of the sum of n1+n2, the sum of n1+n3, and the sum of n1+n4 are greater than 4.

Two additional and separate embodiments provide, respectively, a compound of Formula (VI) and a compound of Formula (VII):

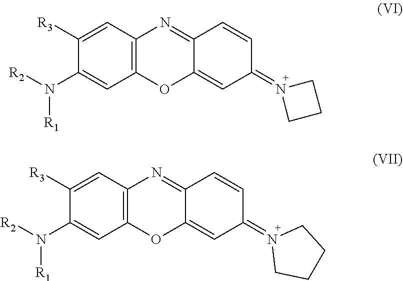

wherein all variables, including R₁, R₂, R₃, X₁, n, n1, n2, n3, and n4, along with all provisos, are as defined for Formula (I), above.

Two more separate embodiments provide, respectively, a compound of Formula (VI) and a compound of Formula (VII), wherein in each embodiment R₃ is H, and R₁ and R₂ are each independently selected from the group of methyl, ethyl, n-propyl, isopropyl, n-butyl, sec-butyl, and tert-butyl.

Two more separate embodiments provide, respectively, a compound of Formula (VI) and a compound of Formula (VII), wherein in each embodiment R₃ is H, and R₁ and R₂ are each independently selected from the group of methyl, ethyl, n-propyl, isopropyl, n-butyl, sec-butyl, and tert-butyl, with the proviso that R₁ and R₂ are not the same.

Two more separate embodiments provide, respectively, a compound of Formula (VI) and a compound of Formula (VII), wherein in each embodiment R₃ is H, and R₁ and R₂ are each independently selected from the group of methyl, ethyl, n-propyl, and isopropyl, with the proviso that R₁ and R₂ are not the same.

Two further separate embodiments provide, respectively, a compound of Formula (VI) and a compound of Formula (VII), wherein in each embodiment R₃ is H, and R₁ is selected from the group of methyl, ethyl, n-propyl, isopropyl, n-butyl, sec-butyl, and tert-butyl, and R₂ is selected from the group of —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:

a)

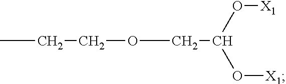

b)

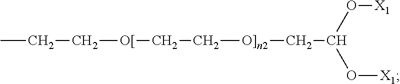

c)

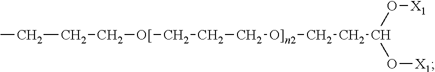

and

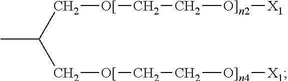

X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;

n1 is an integer independently selected in each instance from the group of 1, 2, 3, and 4;

n2 is an integer independently selected in each instance from the group of 1, 2, 3, 4, 5, 6, 7, 8, 9, and 10;

n3 is an integer independently selected in each instance from the group of 1, 2, 3, 4, 5, 6, 7, 8, 9, and 10;

n4 is an integer independently selected in each instance from the group of 1, 2, 3, 4, 5, 6, 7, 8, 9, and 10; and

with the proviso that the sum of n2+n4 when R₂ is the moiety represented by k), above, is not greater than 10.

A further embodiment comprises a compound of Formula (VIa):

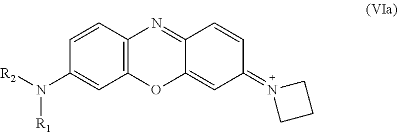

wherein R₁ and R₂ are selected independently from C₁-C₆ alkyl.

Another embodiment provides a compound of Formula (VIa), wherein R₁ and R₂ are each selected independently from C₁-C₄ alkyl.

Another embodiment provides a compound of Formula (VIa), wherein R₁ and R₂ are each selected independently from C₁-C₃ alkyl.

Another embodiment provides a compound of Formula (VIa), wherein R₁ is ethyl and R₂ is C₁-C₄ alkyl.

Another embodiment provides a compound of Formula (VIa), wherein R₁ is ethyl and R₂ is C₁-C₃ alkyl.

Another embodiment provides a compound of Formula (VIa), wherein R₁ is methyl and R₂ is C₁-C₄ alkyl.

Another embodiment provides a compound of Formula (VIa), wherein R₁ is methyl and R₂ is C₁-C₃ alkyl.

Another embodiment provides a compound of Formula (II):

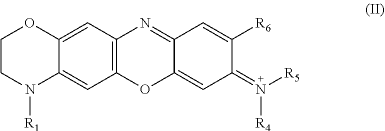

wherein R₁, R₄, R₅, R₆, and all associated variables and provisos are as defined for Formula (I), above.

An additional embodiment provides a compound of Formula (II), above, wherein R1 is as defined above for Formula (I), R₆ is hydrogen, and R₄ and R₅ are each independently selected from the group of straight or branched C₁-C₆ alkyl, with the proviso that, when R₁ is methyl and R₄ is ethyl, R₅ is not ethyl.

A further embodiment provides use of and compositions comprising a compound of Formula (VIII):

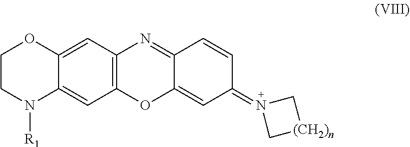

wherein n and R₁, along with all other associated variables and provisos, are as defined for Formula (I), above.

An additional embodiment provides use of and compositions comprising a compound of Formula (VIIIa):

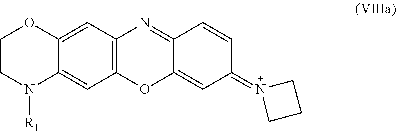

wherein R₁, along with all other associated variables and provisos, are as defined for Formula (I), above.

Another additional embodiment provides use of and compositions comprising a compound of Formula (VIII):

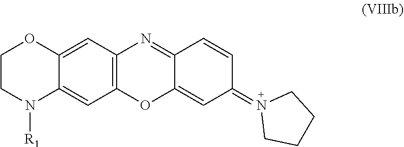

wherein R₁, along with all other associated variables and provisos, are as defined for Formula (I), above.

A still further embodiment provides use of and compositions comprising a compound of Formula (III):

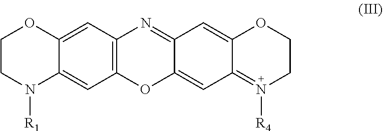

wherein R₁ and R₄, along with all associated variables and provisos, are as defined for Formula (I), above, with the proviso that, when R₁ is methyl, R₄ is not methyl, and with the proviso that, when R₁ is ethyl, R₄ is not ethyl.

Another embodiment provides use of and compositions comprising a compound of Formula (III), above, wherein R₁ is C₁-C₆ alkyl, and R₁, along with all other associated variables and provisos, are as defined for Formula (I), above, and with the proviso that, when R₁ is methyl, R₄ is not methyl, and with the proviso that, when R₁ is ethyl, R₄ is not ethyl.

Still another embodiment provides use of and compositions comprising a compound of Formula (III), above, wherein R₁ is C₁-C₄ alkyl, and R₄, along with all other associated variables and provisos, are as defined for Formula (I), above, and with the proviso that, when R₁ is methyl, R₄ is not methyl, and with the proviso that, when R₁ is ethyl, R₄ is not ethyl.

Still another embodiment provides use of and compositions comprising a compound of Formula (III), above, wherein R₁ is C₁-C₃ alkyl, and R₄, along with all other associated variables and provisos, are as defined for Formula (I), above, and with the proviso that, when R₁ is methyl, R₄ is not methyl, and with the proviso that, when R₁ is ethyl, R₄ is not ethyl.

Still another embodiment provides use of and compositions comprising a compound of Formula (III), above, wherein R₁ is C₁-C₂ alkyl, and R₁, along with all other associated variables and provisos, are as defined for Formula (I), above, and with the proviso that, when R₁ is methyl, R₄ is not methyl, and with the proviso that, when R₁ is ethyl, R₄ is not ethyl.

Yet another embodiment provides use of and compositions comprising a compound of Formula (III), above, wherein R₁ and R₂ are each independently selected from C₁-C₆ alkyl, with the proviso that, when R₁ is methyl, R₄ is not methyl, and with the proviso that, when R₁ is ethyl, R₄ is not ethyl.

A further embodiment provides use of and compositions comprising a compound of Formula (III), above, wherein R₁ and R₂ are each independently selected from C₁-C₄ alkyl, with the proviso that, when R₁ is methyl, R₄ is not methyl, and with the proviso that, when R₁ is ethyl, R₄ is not ethyl.

Another embodiment provides use of and compositions comprising a compound of Formula (III), above, wherein R₁ and R₂ are each independently selected from C₁-C₃ alkyl, with the proviso that, when R₁ is methyl, R₄ is not methyl, and with the proviso that, when R₁ is ethyl, R₄ is not ethyl.

An additional embodiment provides use of and compositions comprising a compound of formula (IX):

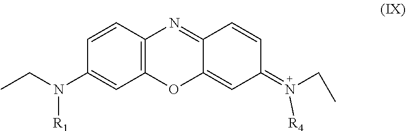

wherein R₁ and R₄ are each independently selected from C₁-C₆ alkyl.

A further embodiment provides use of and compositions comprising a compound of formula (IX), wherein R₁ and R₄ are each independently selected from C₁-C₄ alkyl.

Another embodiment provides use of and compositions comprising a compound of formula (IX), wherein R₁ and R₄ are each independently selected from C₁-C₃ alkyl.

A further embodiment provides use of and compositions comprising a compound of formula (IX), wherein R₁ and R₄ are each independently selected from C₁-C₂ alkyl.

A further embodiment provides use of and compositions comprising a compound of Formula (IX), wherein R₁ is as defined for Formula (I), above, along with all the defined associated variables and provisos.

Gel-Based Formulation for Direct Administration. Gelatin is an animal based protein produced from collagen. The intermolecular bonds in gelatin mainly consist of hydrogen bonds which make the gel thermally reversible. The gel melts below human body temperature making it ideal for injecting it through tubes. Solutions with three concentrations (1.0, 3.0 and 5.0%, w/v) of gelatin were prepared by allowing the gelatin to swell in distilled water overnight (15 h) followed by heating at 45° C. for 30 min to dissolve it. Once in solution, the solubilized dye is spiked into the solution and vortexed. However, the solution was too viscous to let the dye molecule pass through the polymer, leading to low nerve staining.

F127 (Poloxamer 407) has been studied most extensively as a drug delivery system since it has the least toxicity in the commercially available nonionic triblock copolymers composed of a centrail hydrophobic chain of polyoxypropylene flanked by two hydrophilic chains of poly oxyethylene (PLURONIC® series). F127 is more soluble in cold water than in hot water as a result of increased solvation and hydrogen bonding at lower temperatures. At 20% or higher w/w concentration in water, F127 aqueous solutions are liquid at refrigerated temperatures (4-5° C.) but converts to gel upon warming to room temperature. The gelation is reversible upon cooling. This phenomenon suggests that when applied directly or injected into a body cavity or subcutaneously, the gel preparation will form a solid artificial barrier and a sustained release depot. The unique sol-gel-sol transition behavior makes this system a very attractive drug delivery system for painting the tissue of interest. Three different concentrations (20%, 22%, 25%) F127 gels were evaluated and based on animal screening it was found that 22% F127 showed optimum retention at the tissue site with ideal nerve uptake. Particular embodiments can utilize a PEO-PPO-PEO (poly(ethylene oxide)-poly(propylene oxide)-poly(ethylene oxide)) triblock copolymer with a molecular weight of 12,000-13,000 Dalton. Particular embodiments can utilize a PEO-PPO-PEO triblock copolymer with a molecular weight of 12,600 Dalton. In particular embodiments, a formulation including a fluorophore for direct administration can include a concentration of PEO-PPO-PEO triblock copolymer at a range of 18% to 26%, at a range of 19% to 25%, at a range of 20% to 24%, and at a range of 21% to 23%. In particular embodiments, a formulation for direct administration can include a concentration of PEO-PPO-PEO triblock copolymer at 18%, 19%, 20%, 21%, 22%, 23%, 24%, 25%, and 26%, and all values in between. In particular embodiments, a formulation for direct administration can include a concentration of PEO-PPO-PEO triblock copolymer at 22%. In particular embodiments, a gel-based formulation for tissue imaging includes (i) 18-26% PEO-PPO-PEO triblock copolymer; (ii) 19-25% PEO-PPO-PEO triblock copolymer; (iii) 20-24% PEO-PPO-PEO triblock copolymer; and (iv) 21-23% PEO-PPO-PEO triblock copolymer.

F-127 is an FDA approved polymer and its use within the current disclosure is novel because of the following reasons:
i) PEO-PPO-PEO triblock based gel has never been used to paint a tissue site of interest.
ii) The formulation increases solubility as well as permeability of a nerve-specific fluorophore.
iii) The formulation provides ideal viscosity for maximum retention of the nerve-specific fluorophore on the site of application without running out.
iv) The formulation produces similar nerve to muscle ratio as the co-solvent formulation, which being toxic is not applicable in clinic.

Thus, disclosed herein is the development of a clinically viable gel-based formulation strategy that enables direct administration of nerve-specific fluorescent contrast agents with increased control for nerve sparing FGS applications. A PEO-PPO-PEO triblock (e.g., F127 Pluronic®) formulation was used to solubilize a novel near-infrared nerve specific oxazine fluorophore, LGW01-08, providing increased staining control for a variety of tissue surfaces, angles, and morphologies. Additionally, the formulation developed herein possesses unique gelling characteristics, allowing it to easily be spread as a liquid followed by rapid gelling at body temperature for subsequent tissue hold. Further analysis of the direct administration protocol has decreased the time to complete staining to a total of 1-2 minutes. The resulting gel formulation and direct administration methodology provides an ideal platform for clinical translation of novel nerve-specific fluorophores for FGS.

Alginates have been used as versatile bio-polymers for numerous applications. Alginates have conventionally been used as thickening, gel-forming and stabilizing agents. Alginate has a unique property of gelling in the presence of divalent ions such as calcium. 5.0%, 6.8% and 8% were evaluated and when screened for maximum retention at the application site, 6.8% was ideal but compared to PEO-PPO-PEO triblock it performed poorly in terms of the desired retention at the tissue site. In particular embodiments, a formulation including a fluorophore for direct administration can include a concentration of sodium alginate at a range of 5% to 10%, at a range of 5% to 9%, at a range of 5% to 8%, at a range of 6% to 8%, and at a range of 6% to 7%. In particular embodiments, a formulation for direct administration can include a concentration of sodium alginate at 5%, 6%, 7%, 8%, 9%, and 10%, and all values in between. In particular embodiments, a formulation for direct administration can include a concentration of sodium alginate at 6.5%. In particular embodiments, a gel-based formulation for tissue imaging includes (i) 5-10% sodium alginate; (ii) 5-9% sodium alginate; (iii) 5-8% sodium alginate; (iv) 6-8% sodium alginate; and (v) 6-7% sodium alginate. In particular embodiments, a formulation including a fluorophore for direct administration can include 5-10% alginate salt. In particular embodiments, an alignate salt includes calcium alginate, potassium alginate, magnesium alginate, and ammonium alginate.

Also provided is a gel-based formulation for tissue imaging including (i) a fluorophore and (ii) 5-8% sodium alginate and/or 20-24% PEO-PPO-PEO block copolymer.

Further provided is a gel-based formulation for tissue imaging including (i) a fluorophore and (ii) 6-7% sodium alginate and/or 20-24% PEO-PPO-PEO block copolymer.

Further provided is a gel-based formulation for tissue imaging including (i) a fluorophore and (ii) 6-7% sodium alginate and/or 21-23% PEO-PPO-PEO block copolymer.

Accordingly, a PEO-PPO-PEO triblock based formulation and/or a sodium alginate based formulation strategy is preferred to replace the laboratory grade co-solvent formulation used for initial development of nerve-specific fluorophores described herein.

Systemic Administration. To maintain nerve-specificity, the molecular weight of fluorophore must remain low enough to permit passing through the blood-nerve-barrier following systemic administration. Thus, although LGW01-08 has demonstrated high nerve specificity and adequate fluorescence signal for real time imaging following systemic administration, the fluorophore's lipophilicity necessitates formulation as a vehicle for intravenous injection. Gibbs-Strauss et al. Molecular imaging 10, 91-101 (2011); Barth & Gibbs. Theranostics 7, 573-593 (2017).

Formulations for Systemic Administration. Based on the solubility of the nerve specific dye, liposomes sphingomyelin cholesterol (55:45), a FDA approved lipid mix was considered first for encapsulation of the agent as the lipid vesicles have both the lipid bilayer and hydrophilic core. Two common methods for preparation of liposomes are passive and pH gradient method. For passive loading method, both LGW01-08 and lipids were dissolved in a mixture of chloroform and methanol (70:30) and subjected to rotary evaporation under vacuum. Upon achieving a thin film, the film was hydrated in citrate buffer at 200 rpm at 60° C. for an hour, after which the solution was extruded at 60° C. using Avanti extruder for 20 passes to obtain unilamellar vesicles. The vesicles were then passed through a column to separate the unencapsulated dye and stored at 4° C. till further use. However, as expected poor loading was achieved with the passive loading method.

To achieve better loading, pH gradient was achieved by using ammonium sulphate buffer instead of citrate buffer and the internal pH of the vesicle was made acidic by passing the lipid solution through a column pre-equilibrated with HEPES buffer pH 7.4. This method increased the loading to 0.26 mg/mL, which was around the solubility of LGW01-08 (0.25 mg/mL). Thus, liposomal formulation was not successful in increasing the solubility of LGW01-08.

The next delivery platform which could increase the solubility of the nerve dye without compromising the nerve specificity was micelles. They are 10-100 nm in size and include a core and shell structure; the inner core is the hydrophobic part of the block copolymer, which encapsulates the poorly water-soluble drug, whereas the outer shell formed by the hydrophilic block of the copolymer protects the drug from the aqueous environment and stabilizes the micelle against recognition in vivo by the reticuloendothelial system. Commercially available (A-B-A block), PLURONIC® ethylene oxide-propylene oxide block co-polymer capable of self-assembly to form micelle was evaluated for encapsulating the nerve dye. The size and hydrophobic blocks in the copolymer influence the loading of the molecule in the micelles. Based on previous experience, the PEO-PPO-PEO triblock was weighed and dissolved in acetone to which the dye pre-dissolved in acetone was added. The mixture was subjected to rotary evaporation under vacuum. Upon achieving a thin film, the film was hydrated in saline and centrifuged at 7500 g for 10 minutes. The supernatant was passed through a 0.22μ filter and analyzed for nerve dye loading. The solubility was increased almost 2-fold and a encapsulation of 0.46 mg/mL was achieved.

In order to achieve higher encapsulation, two more polymeric micelle platforms: Poly(ethylene glycol)(2k)-block-poly(D,L-lactic acid) (1.8k) (PEG-b-PLA) and Distearyl-phosphatidylethanolamine-PEG2000 (DSPE-PEG) were evaluated. Both the micelles were prepared using the solvent casting method. Briefly, 2 mg of LGW01-08 and 60 mg of DSPE-PEG lipid/60 mg PEG-PLA dissolved in 2 mL acetonitrile were combined in a 25 mL round bottom flask, which was evaporated under reduced pressure to form a thin dye distributed polymeric lipid film. The film was rehydrated in 2 mL of normal saline water by vortexing, centrifuged and filtered using a 0.22 micron nylon filter. Micelles were characterized for drug loading by using multiskan spectrum spectrophotometer (Thermo Fischer, Waltham, Mass.). Triplicate samples were prepared for quantification by diluting the complex 100-fold in 10% triton solution. The encapsulation achieved for DSPE-PEG micelle was higher than the PEG-PLA micelles. The solubility of the nerve dye was increased almost 3 folds in the DSPE-PEG micelles.

The goal was to have multiple formulations for animal nerve specificity screening and thus one additional FDA approved polymer, hydroxy-propyl-β cyclodextrin (HP-β-CD) was evaluated for loading of the nerve dye. Cyclodextrin can enhance the aqueous solubility of poorly water soluble molecules and at the same time increase the ability to permeate through biological membranes. Particularly, hydroxypropyl derivatives have shown to increase solubility, stability and bioavailability of numerous molecules.

HP-β-CD complex was prepared by the solvent casting method. Briefly, 2 mg of LGW01-08 and 60 mg of HP-β-CD was dissolved in 2 mL of 95% ethanol, which was evaporated under reduced pressure to form a thin dye distributed HP-β-CD film. The HP-β-CD complex was achieved by rehydration of the thin film with normal saline, centrifuged and filtered through 0.22 micron nylon filter. The LGW01-08 loading in the complex was quantified by photometric analysis at 638 nm using multiskan spectrum spectrophotometer (Thermo Fischer, Waltham, Mass.). Triplicate samples were prepared for quantification by diluting the complex 100-fold in 10% triton solution.

In particular embodiments, a formulation for systemic administration includes a fluorophore encapsulated by DSPE-PEG micelles at a concentration of 0.5 mg/mL to 0.9 mg/mL, 0.5 mg/mL to 1.2 mg/mL, or 0.7 mg/mL to 1.0 mg/mL In particular embodiments, a formulation for systemic administration includes a fluorophore encapsulated by DSPE-PEG micelles at 0.5 mg/mL, 0.6 mg/mL, 0.7 mg/mL, 0.8 mg/mL, 0.9 mg/mL, 1.0 mg/mL, 1.1 mg/mL, and 1.2 mg/mL In particular embodiments, a formulation for systemic administration includes a fluorophore encapsulated by DSPE-PEG micelles at 0.7 mg/mL In particular embodiments, a formulation for systemic administration includes a fluorophore encapsulated by cyclodextrin at a concentration of 0.5 mg/mL to 0.9 mg/mL, 0.5 mg/mL to 1.2 mg/mL, or 0.7 mg/mL to 1.0 mg/mL In particular embodiments, a formulation for systemic administration includes a fluorophore encapsulated by cyclodextrin at 0.5 mg/mL, 0.6 mg/mL, 0.7 mg/mL, 0.8 mg/mL, 0.9 mg/mL, 1.0 mg/mL, 1.1 mg/mL, and 1.2 mg/mL. In particular embodiments, a formulation for systemic administration includes a fluorophore encapsulated by cyclodextrin at 0.7 mg/mL.

Both a cyclodextrin and DSPE-PEG micelle-based formulation strategy are preferred to replace the laboratory grade co-solvent formulation used for initial nerve-specific fluorophore development. Both of these formulation strategies are FDA approved and are novel because of the following reasons:
v) Both increase the solubility of nerve specific dye
vi) They produce similar nerve to muscle ratio as the co-solvent formulation, which being toxic is not applicable in clinic.
vii) They do not modify the pharmacokinetic property of the dye resulting in circulation half-life of the dye to be around 12 h, ideal for the clinical application of the molecule.

The disclosed formulations for delivery of fluorophores have improved stability compared to previously employed co-solvent formulations. Additionally, toxicity testing, pharmacokinetics, and dose ranging studies have been carried out to determine important pharmacological properties of the novel fluorophore/formulation combinations. The work presented herein provides an extensive framework for clinical translation of this promising nerve specific contrast agent to improve nerve and tissue identification intraoperatively.

Chemical Synthesis.

Chemical Synthesis

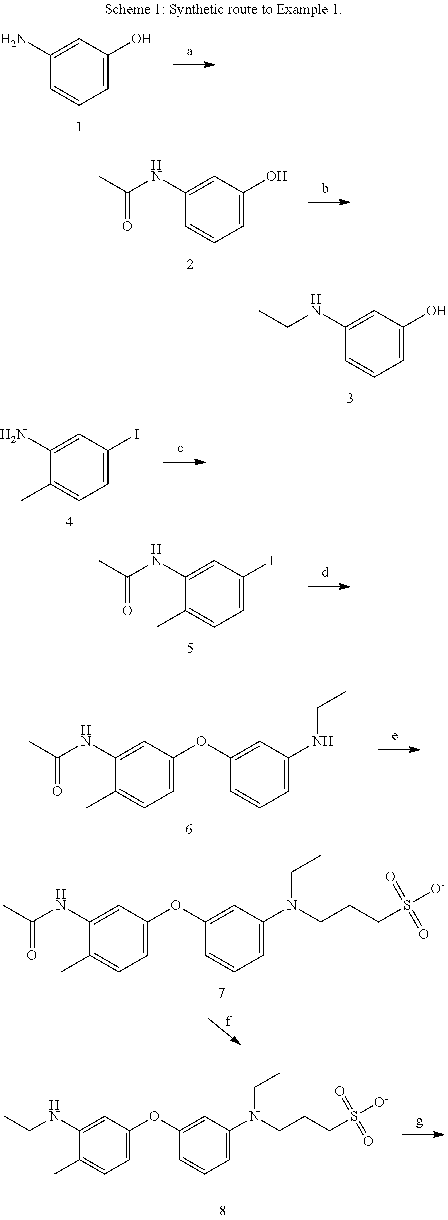

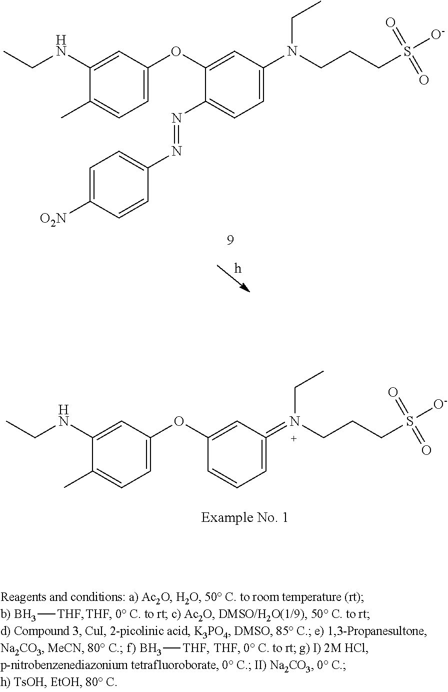

### EXAMPLE NO. 1

### (E)-3-(ethyl(7-(ethylamino)-8-methyl-3H-phenoxazin-3-ylidene)ammonio)propane-1-sulfonate

N-(3-hydroxyphenyl)acetamide (2): Compound 1 (1 g, 9.16 mmol) was suspended in 10 mL DI water, to which Acetic anhydride (2.60 mL, 27.49 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of ice-cold DI water. The product was left in the funnel and air dried overnight to afford compound 2 (1.19 g, 86%) as a light gray solid, which was used for the next step without further purification.

3-(ethylamino)phenol (3): A solution of 2 (1 g, 6.62 mmol) in anhydrous THF (20 mL) was stirred in an ice bath under N₂ for 30 mins. Borane tetrahydrofuran complex solution (1 M, 20 mL) was added to the solution above using a syringe pump over 30 mins, while maintaining the temperature of the solution below 5° C. The resulting reaction mixture was left in the ice bath and slowly warmed to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel (25 g), using DCM/Hexane as eluent to obtain 3 (832 mg, 92%) as a solid.

N-(5-iodo-2-methylphenyl)acetamide (5): Compound 4 (2 g, 8.58 mmol) was dissolved in 2 mL DMSO, to which Acetic anhydride (2.43 mL, 25.75 mmol) was added dropwise. The reaction mixture was stirred in a water bath (50° C.) for 10 min, then stirred for additional 2 h at rt. 18 mL DI water was added to the reaction mixture, the resulting suspension was stirred overnight at rt. The solid was then collected via vacuum filtration and washed with small portions of DI water. The product was left in the funnel and air dried to afford compound 5 (2.09 g, 89%) as a light gray solid, which was used for the next step without further purification.

N-(5-(3-(ethylamino)phenoxy)-2-methylphenyl)acetamide (6): Compound 6 was synthesized using a slightly modified protocol published by Maiti and Buchwald.² An oven-dried microwave glass tube was charged with a magnetic stir bar, compound 3 (500 mg, 3.64 mmol), compound 5 (1.05 g, 3.83 mmol), CuI (69 mg, 0.36 mmol), 2-picolinic acid (90 mg, 0.73 mmol), and anhydrous K₃PO₄ (1.55 g, 7.29 mmol). The glass tube was evacuated under vacuum and backfilled 5 times with N₂ before the tube was immediately sealed with a Teflon cap. Anhydrous DMSO (5 mL) was delivered via a syringe. The reaction was then heated to 85° C. and stirred for 18 h. After cooling to rt, the reaction mixture was diluted with 50 mL DI water and extracted with DCM (4×50 mL). The combined organic layers were washed with brine and dried over anhydrous Na₂SO₄, then concentrated in vacuo. The residue was purified by flash column chromatography with silica gel (25 g), using DCM/Hexane as eluent to give compound 6 (871 mg, 74%) as an orange oil.

3-((3-(3-acetamido-4-methylphenoxy)phenyl)(ethyl)amino)propane-1-sulfonate (7): To a suspension of compound 6 (350 mg, 1.23 mmol) and Na₂CO₃ (261 mg, 2.46 mmol) in anhydrous MeCN (10 mL) under N₂, was added 1,3-Propanesultone (226 mg, 1.85 mmol) at rt. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through celite. The filtrate was concentrated to dryness by a rotary evaporator to give compound 7 (392 mg, 78%), and was used for the next step without further purification.

3-(ethyl(3-(3-(ethylamino)-4-methylphenoxy)phenyl)amino)propane-1-sulfonate (8): A solution of compound 7 (375 mg, 0.923 mmol) in anhydrous THF (10 mL) was stirred in an ice bath under N₂ for 30 mins. Borane tetrahydrofuran complex solution (1 M, 5 mL) was added to the solution above dropwise, while the temperature of the solution was maintained below 5° C. The resulting reaction mixture was left to stir in the ice bath and slowly warm to rt. After 24 h, the solution was placed in an ice bath again, and excess borane reagent was destroyed by carefully adding MeOH until no gas evolved. The solvent was evaporated under reduced pressure to give compound 8 (quantitative) and was used for the next step without further purification.

(E)-3-(ethyl(3-(3-(ethylamino)-4-methylphenoxy)-4-((4-nitrophenyl)diazenyl)phenyl)amino)propane-1-sulfonate (9): Compound 8 (100 mg, 0.255 mmol) was dissolved in MeOH (1 mL). The solution was chilled in an ice bath, then was treated with HCl (2 M, 5 mL). After 15 mins, p-nitrobenzenediazonium tetrafluoroborate (64 mg, 0.268 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C18 cartridge, washed with DI water, air-dried, followed by elution with MeOH. The solvent was evaporated under reduced pressure to give compound 9 (quantitative) and was used for the next step without further purification.

(E)-3-(ethyl(7-(ethylamino)-8-methyl-3H-phenoxazin-3-ylidene)ammonio)propane-1-sulfonate (Example 1): Under N₂, compound 9 (50 mg, 0.092 mmol) and treated with p-toluenesulfonic acid monohydrate (53 mg, 0.277 mmol) were dissolved in ethanol (5 mL). The resulting solution was heated to 80° C., and stirred overnight. The solvent was evaporated under reduced pressure, and the residue was purified by reverse phase HPLC (MeCN/H₂O, 5-50%, linear gradient, TFA 0.1% as additive) to afford the compound of Example 1 (21 mg, 56%) as a dark blue solid.

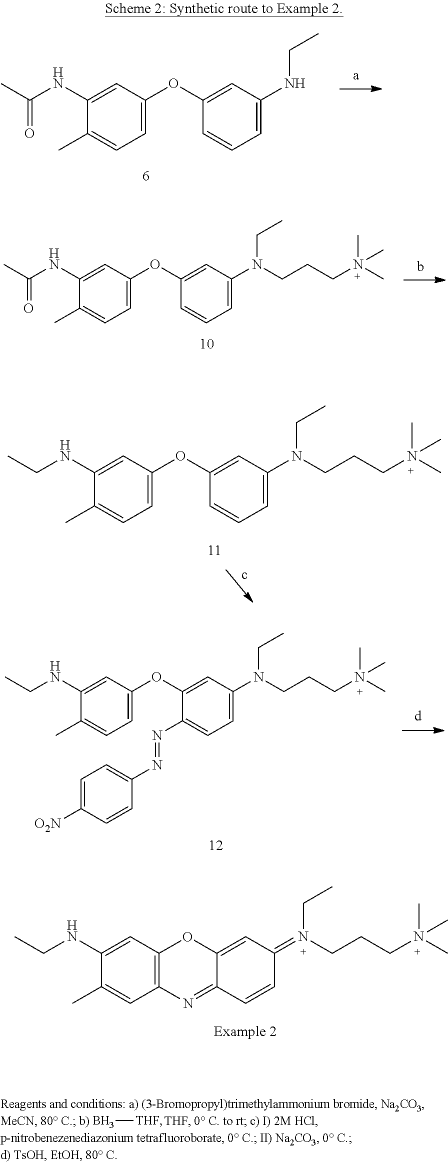

### EXAMPLE NO

### (E)-N¹-ethyl-N¹-(7-(ethylamino)-8-methyl-3h-phenoxazin-3-ylidene)-N³,N³,N³-trimethylpropane-1,3-diaminium

3-((3-(3-acetamido-4-methylphenoxy)phenyl)(ethyl)amino)-N,N,N-trimethylpropan-1-aminium (10): Compound 6 (350 mg, 1.23 mmol), (3-Bromopropyl)trimethylammonium bromide (482 mg, 1.85 mmol), and Na₂CO₃ (261 mg, 2.46 mmol) were suspended in anhydrous MeCN (10 mL) under N₂. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through Celite. The filtrate was concentrated to dryness by a rotary evaporator to give compound 10 (quantitative) and was used for the next step without further purification.

3-(ethyl(3-(3-(ethylamino)-4-methylphenoxy)phenyl)amino)-N,N,N-trimethylpropan-1-aminium (11): A solution of compound 10 (350 mg, 0.91 mmol) in anhydrous THF (10 mL) was stirred in an ice bath under N₂ for 30 mins. Borane tetrahydrofuran complex solution (1 M, 10 mL) was added to the solution above dropwise, while the temperature of the solution was maintained below 5° C. The resulting reaction mixture was left to stir in the ice bath and slowly warm to rt. After 24 h, the solution was placed in an ice bath again, and excess borane reagent was destroyed by carefully adding MeOH until no gas evolved. The solvent was evaporated under reduced pressure to give compound 11 (quantitative) and was used for the next step without further purification.

(E)-3-(ethyl(3-(3-(ethylamino)-4-methylphenoxy)-4-((4-nitrophenyl)diazenyl)phenyl)amino)-N,N,N-trimethylpropan-1-aminium (12): Compound 11 (100 mg, 0.270 mmol) was dissolved in MeOH (1 mL). The solution was chilled in an ice bath, then was treated with HCl (2 M, 5 mL). After 15 mins, p-nitrobenzenediazonium tetrafluoroborate (67 mg, 0.283 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C18 cartridge, washed with DI water, air-dried, followed by elution with MeOH. The solvent was evaporated under reduced pressure to give compound 12 (quantitative) and was used for the next step without further purification.

(E)-N¹-ethyl-N¹-(7-(ethylamino)-8-methyl-3H-phenoxazin-3-ylidene)-N³,N³,N³-trimethylpropane-1,3-diaminium (Example 2): Under N₂, compound 12 (50 mg, 0.096 mmol) and treated with p-toluenesulfonic acid monohydrate (55 mg, 0.289 mmol) were dissolved in ethanol (5 mL). The resulting solution was heated to 80° C., and stirred overnight. The solvent was evaporated under reduced pressure, and the residue was purified by reverse phase HPLC (MeCN/H₂O, 5-50%, linear gradient, TFA 0.1% as additive) to afford Example 2 (33.7 mg, 92%) as a dark blue solid.

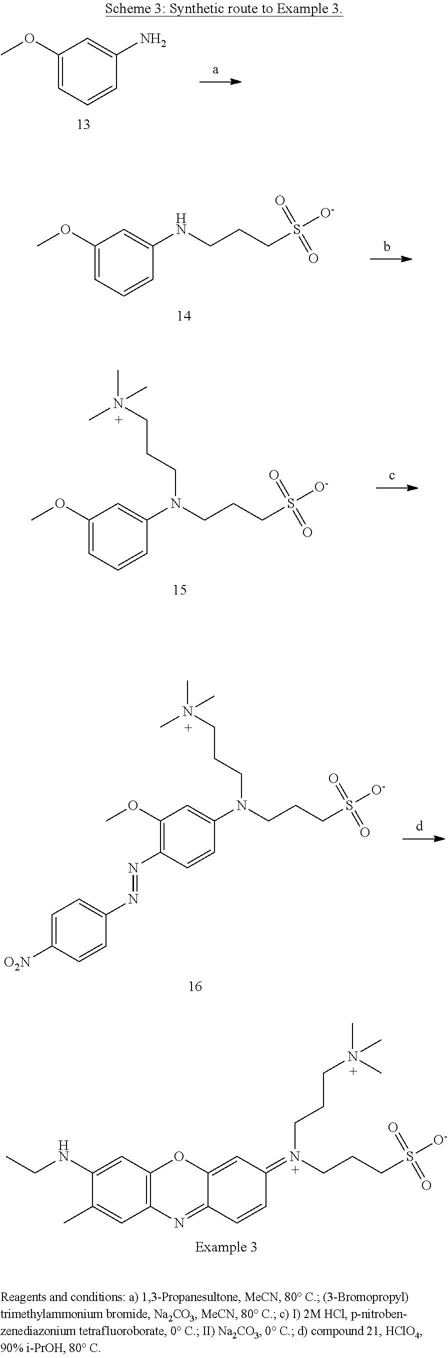

3-((3-methoxyphenyl)amino)propane-1-sulfonate (14): To a suspension of compound 13 (2 g, 16.2 mmol) in anhydrous MeCN (20 mL) under N₂, was added 1,3-Propanesultone (2.02 g, 16.6 mmol) at rt. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through Celite. The filtrate was concentrated to dryness by a rotary evaporator to give compound 14 (3.74 g, 94%), and was used for the next step without further purification.

3-((3-methoxyphenyl)(3-(trimethylammonio)propyl)amino)propane-1-sulfonate (15): Compound 14 (2 g, 8.15 mmol), (3-Bromopropyl)trimethylammonium bromide (1.51 g, 8.32 mmol), and Na₂CO₃ (907 mg, 8.56 mmol) were suspended in anhydrous MeCN (50 mL) under N₂. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through Celite. The filtrate was concentrated to dryness by a rotary evaporator to give compound 15 (quantitative) and was used for the next step without further purification.

(E)-3-((3-methoxy-4-((4-nitrophenyl)diazenyl)phenyl)(3-(trimethylammonio)propyl)amino)propane-1-sulfonate (16): Compound 15 (500 mg, 1.36 mmol) was dissolved in MeOH (2 mL). The solution was chilled in an ice bath, then was treated with HCl (2 M, 20 mL). After 15 mins, p-nitrobenzenediazonium tetrafluoroborate (339 mg, 1.43 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C18 cartridge, washed with DI water, air-dried, followed by elution with MeOH. The solvent was evaporated under reduced pressure to give compound 16 (quantitative) and was used for the next step without further purification.

(E)-3-((7-(ethylamino)-8-methyl-3H-phenoxazin-3-ylidene)(3-(trimethylammonio)propyl) ammonio)propane-1-sulfonate (Example 3): Compound 21 (50 mg, 0.304 mmol) was dissolved in a solution of i-PrOH/H₂O (9:1, 5 mL) at 80° C. for 30 min. Compound 16 (150 mg, 0.304 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure, and the residue was purified by reverse phase HPLC (MeCN/H₂O, 5-50%, linear gradient, TFA 0.1% as additive) to afford the compound of Example No. 3 (71 mg, 49%) as a dark blue solid.

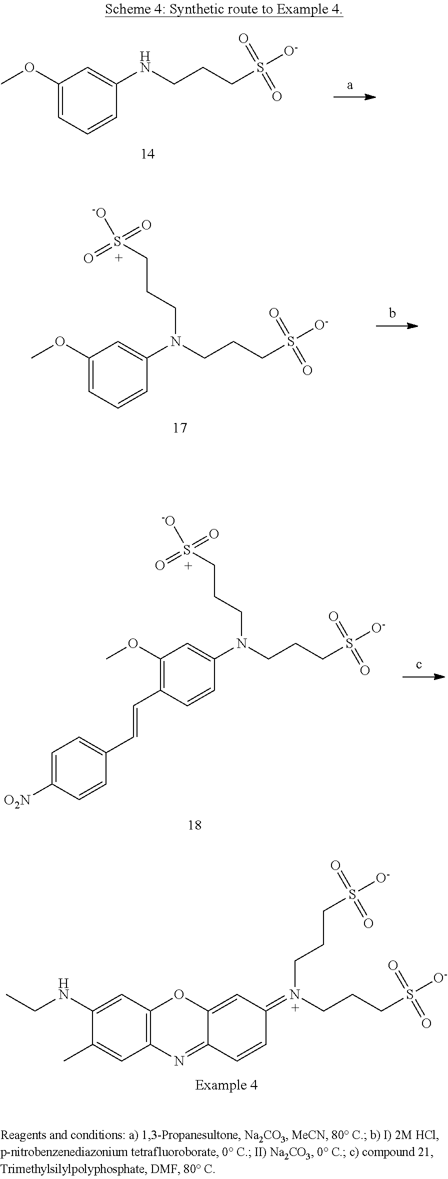

### EXAMPLE NO. 4

### 3,3′-((7-(ethylamino)-8-methyl-3H-phenoxazin-3-ylidene)ammonio)bis(propane-1-sulfonate)

3,3′-((3-methoxyphenyl)azanediyl)bis(propane-1-sulfonate) (17): To a suspension of compound 14 (1 g, 4.08 mmol) and Na₂CO₃ (454 mg, 4.28 mmol) in anhydrous MeCN (20 mL) under N₂, was added 1,3-Propanesultone (508 mg, 4.16 mmol) at rt. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through celite. The filtrate was concentrated to dryness by a rotary evaporator, and the residue was purified by reverse phase HPLC (MeCN/H₂O, 5-50%, linear gradient, TFA 0.1% as additive) to afford compound 17 (1.39 g, 92%).

(E)-3,3′-((3-methoxy-4-((4-nitrophenyl)diazenyl)phenyl)azanediyl)bis(propane-1-sulfonate) (18): Compound 17 (610 mg, 1.66 mmol) was dissolved in MeOH (2 mL). The solution was chilled in an ice bath, then was treated with HCl (2 M, 20 mL). After 15 mins, p-nitrobenzenediazonium tetrafluoroborate (413 mg, 1.74 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C18 cartridge, washed with DI water, air-dried, followed by elution with MeOH. The solvent was evaporated under reduced pressure to give compound 18 (quantitative), and was used for the next step without further purification.

3,3′-((7-(ethylamino)-8-methyl-3H-phenoxazin-3-ylidene)ammonio)bis(propane-1-sulfonate) (Example 4): Compound 21 (5 mg, 0.0304 mmol) and 18 (15.7 mg, 0.0304 mmol) were dissolved DMF (0.5 mL), to which Trimethylsilylpolyphosphate (10 μL) was added. The resulting solution was heated at 80° C. overnight. The crude product was then directly purified by reverse phase HPLC (MeCN/H₂O, 5-50%, linear gradient, TFA 0.1% as additive) to afford the compound of Example 4.

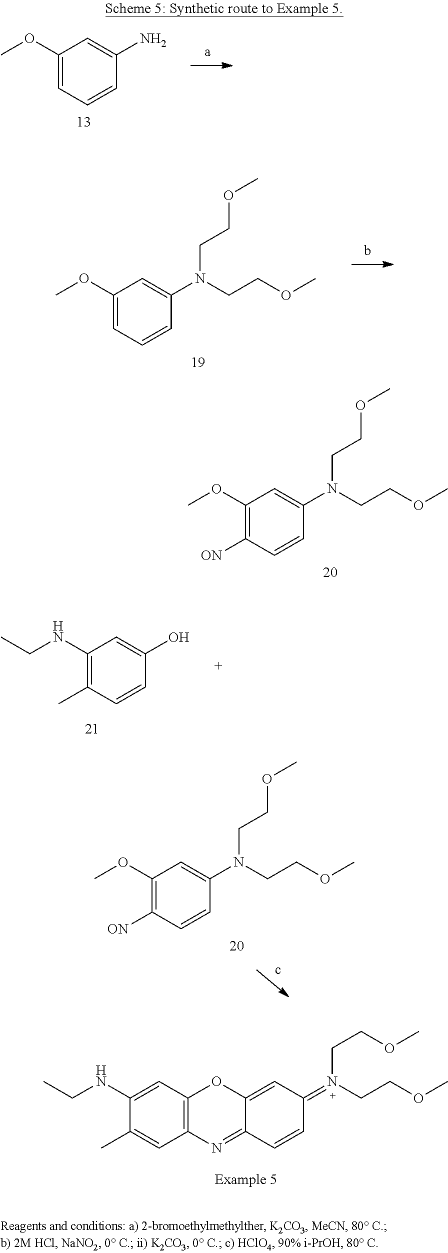

### EXAMPLE NO. 5

### N-(7-(ethylamino)-8-methyl-3H-phenoxazin-3-ylidene)-2-methoxy-N-(2-methoxyethyl)ethan-1-aminium

3-methoxy-N,N-bis(2-methoxyethyl)aniline (19): Compound 13 (1 g, 8.12 mmol), 2-bromoethylmethylther (3.42 g, 24.36 mmol), and K₂CO₃ (2.24 g, 16.2 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel (25 g), using DCM/Hexane as eluent to give compound 19 (1.62 g, 83%) as clear oil.

3-methoxy-N,N-bis(2-methoxyethyl)-4-nitrosoaniline (20): Compound 19 (1.2 g, 5.01 mmol) was dissolved in an ice-cold 2 M HCl solution (15 mL). To this solution was added NaNO₂ (381 mg, 5.52 mmol) portion-wise over 1 h while the temperature of the solution was maintained below 5° C., such that no brown NOx vapors were observed. The reaction mixture was stirred for an additional 2 h. The solution was carefully basified with solid K₂CO₃ until the pH value of the solution had risen above 8. The resulting precipitate was filtered through a Büchner funnel and washed with small portions of ice-cold DI water. The title compound was obtained (1.03 g, 77%) as a green solid, which was used for the next step without further purification.

N-(7-(ethylamino)-8-methyl-3H-phenoxazin-3-ylidene)-2-methoxy-N-(2-methoxyethyl)ethan-1-aminium (Example 5): Compound 21 (50 mg, 0.304 mmol) was dissolved in a solution of i-PrOH/H₂O (9:1, 5 mL) at 80° C. for 30 min. Compound 20 (86 mg, 0.319 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH containing 0.5% formic acid to give compound of Example 5 (62 mg, 55%).

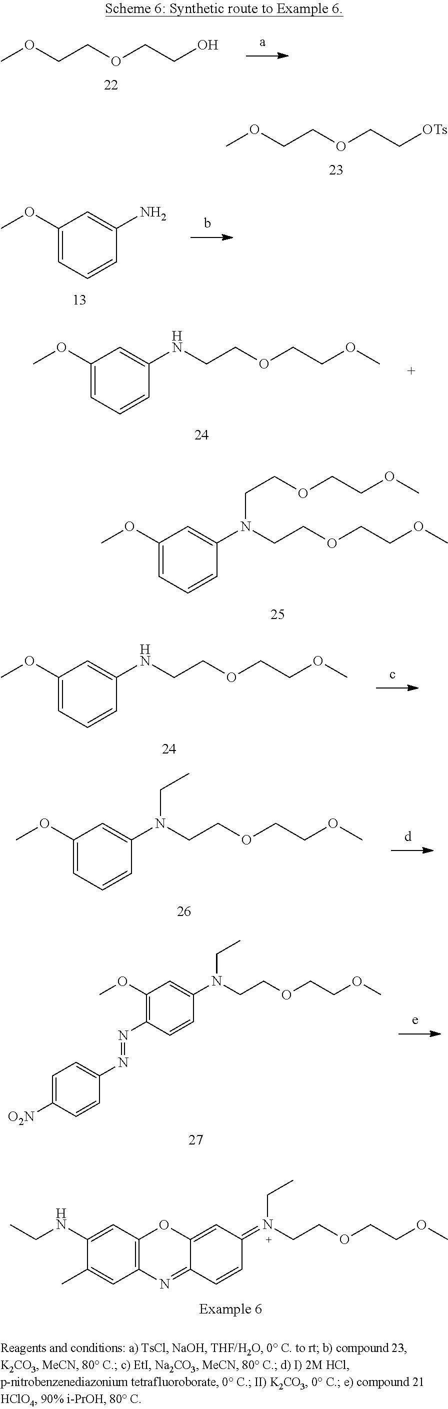

### EXAMPLE NO. 6

2-(2-methoxyethoxy)ethyl 4-methyl benzenesulfonate (23): To a THF solution (25 mL) of diethylene glycol methyl ether (5 g, 41.6 mmol) was added NaOH (20%, 25 mL). The resulting solution was chilled in an ice bath before TsCl (9.52 g, 49.9 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

3-methoxy-N-(2-(2-methoxyethoxy)ethyl)aniline (24) and 3-methoxy-N,N-bis(2-(2-methoxyethoxy)ethyl)aniline (25): Compound 13 (1 g, 8.12 mmol), compound 23 (4.46 g, 16.2 mmol), and K₂CO₃ (2.24 g, 16.2 0.798 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel (25 g), using DCM/Hexane as eluent to give compound 24 (0.618 g, 34%) and 25 (1.06 g, 40%).

N-ethyl-3-methoxy-N-(2-(2-methoxyethoxy)ethyl)aniline (26): Compound 24 (500 mg, 2.22 mmol), EtI (363 mg, 2.33 mmol), and Na₂CO₃ (353 mg, 3.33 mmol) were suspended in anhydrous MeCN (20 mL) under N2. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel (25 g), using DCM/Hexane as eluent to give compound 26 (449 g, 80%) as a clear oil.

(E)-N-ethyl-3-methoxy-N-(2-(2-(2-methoxyethoxy)ethoxy)ethyl)-4-((4-nitrophenyl)diazenyl)aniline (27): Compound 26 (300 mg, 1.18 mmol) was dissolved in MeOH (2 mL). The solution was chilled in an ice bath, then was treated with HCl (2 M, 20 mL). After 15 mins, p-nitrobenzenediazonium tetrafluoroborate (295 mg, 1.24 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

(E)-N-ethyl-N-(7-(ethylamino)-8-methyl-3H-phenoxazin-3-ylidene)-2-(2-methoxyethoxy)ethan-1-aminium (Example 6): Compound 21 (50 mg, 0.304 mmol) was dissolved in a solution of i-PrOH/H₂O (9:1, 5 mL) at 80° C. for 30 min. Compound 27 (122 mg, 0.304 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 6 (69 mg, 59%).

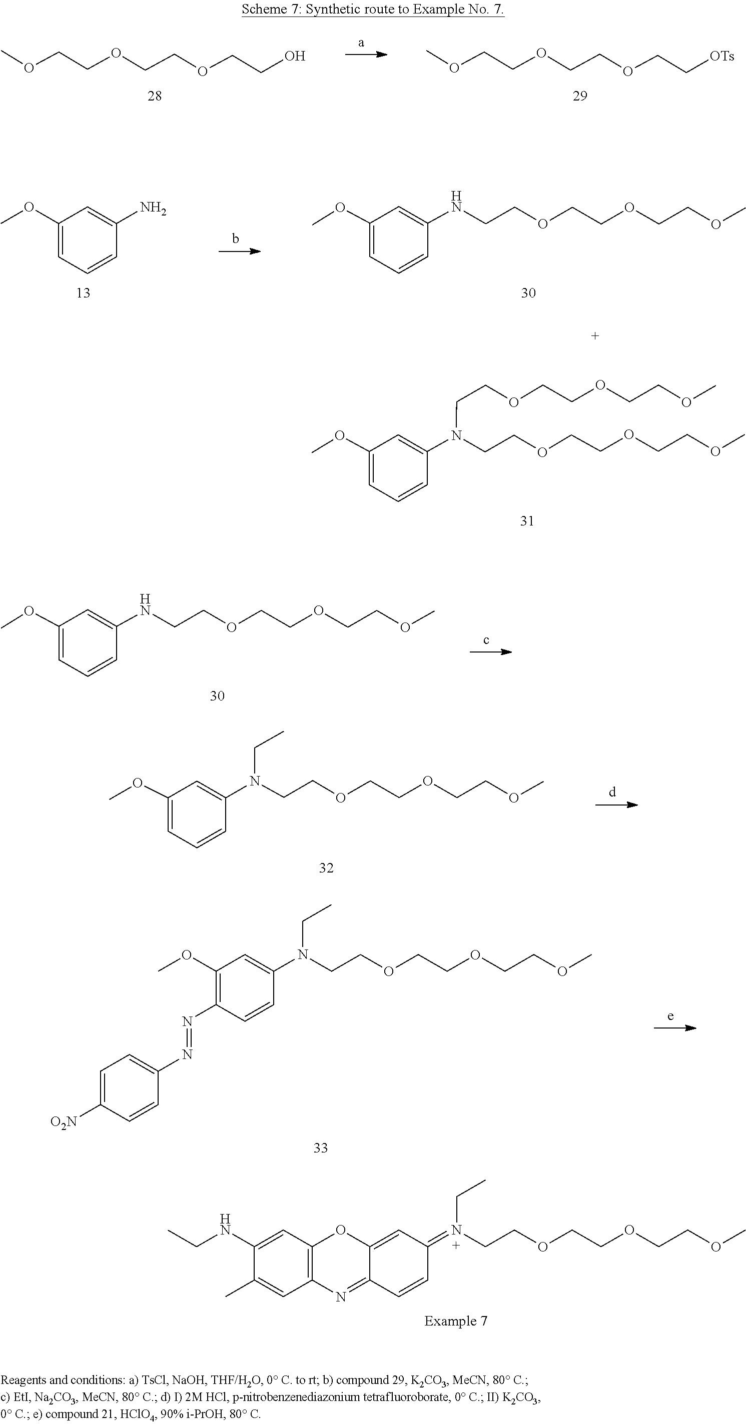

### EXAMPLE NO. 7

### (E)-N-ethyl-N-(7-(ethylamino)-8-methyl-3H-phenoxazin-3-ylidene)-2-(2-(2-methoxyethoxy)ethoxy)ethan-1-aminium

2-(2-(2-methoxyethoxy)ethoxy)ethyl 4-methylbenzenesulfonate (29): To a THF solution (25 mL) of Triethylene glycol monomethyl ether (5 g, 30.5 mmol) was added NaOH (20%, 25 mL). The resulting solution was chilled in an ice bath before TsCl (6.97 g, 36.5 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

3-methoxy-N-(2-(2-(2-methoxyethoxy)ethoxy)ethyl)aniline (30) and 3-methoxy-N,N-bis(2-(2-(2-methoxyethoxy)ethoxy)ethyl)aniline (31): Compound 13 (500 mg, 4.06 mmol), compound 29 (5.17 g, 16.2 mmol), and K₂CO₃ (2.24 g, 16.2 mmol) were suspended in anhydrous MeCN (10 mL) under N₂. The reaction mixture was then heated to 80° C. and stirred for 24 h before diluted with DCM (25 mL). The solid was removed via vacuum filtration through Celite. The solvent was removed using a rotary evaporator and the residue was purified by flash column chromatography with silica gel (25 g), using DCM/Hexane as eluent to give compound 30 (458 g, 42%) and 31 (0.601 g, 36%).

N-ethyl-3-methoxy-N-(2-(2-(2-methoxyethoxy)ethoxy)ethyl)aniline (32): Compound 30 (400 mg, 1.49 mmol), EtI (236 mg, 1.51 mmol), and Na₂CO₃ (165 mg, 1.56 mmol) were suspended in anhydrous MeCN (10 mL) under N₂. The reaction mixture was then heated to 80° C. and stirred for 24 h before diluted with DCM (25 mL). The solid was removed via vacuum filtration through Celite. The solvent was removed using a rotary evaporator and the residue was purified by flash column chromatography with silica gel (25 g), using DCM/Hexane as eluent to give compound 32 (376 mg, 85%).

(E)-N-ethyl-3-methoxy-N-(2-(2-(2-methoxyethoxy)ethoxy)ethyl)-4-((4-nitrophenyl)diazenyl)aniline (33): Compound 32 (300 mg, 1.01 mmol) was dissolved in MeOH (2 mL). The solution was chilled in an ice bath, then was treated with HCl (2 M, 20 mL). After 15 mins, p-nitrobenzenediazonium tetrafluoroborate (251 mg, 1.06 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

(E)-N-ethyl-N-(7-(ethylamino)-8-methyl-3H-phenoxazin-3-ylidene)-2-(2-(2-methoxyethoxy)ethoxy)ethan-1-aminium (Example 7): Compound 21 (50 mg, 0.304 mmol) was dissolved in a solution of i-PrOH/H₂O (9:1, 5 mL) at 80° C. for 30 min. Compound 33 (136 mg, 0.304 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 7 (53 mg, 41%).

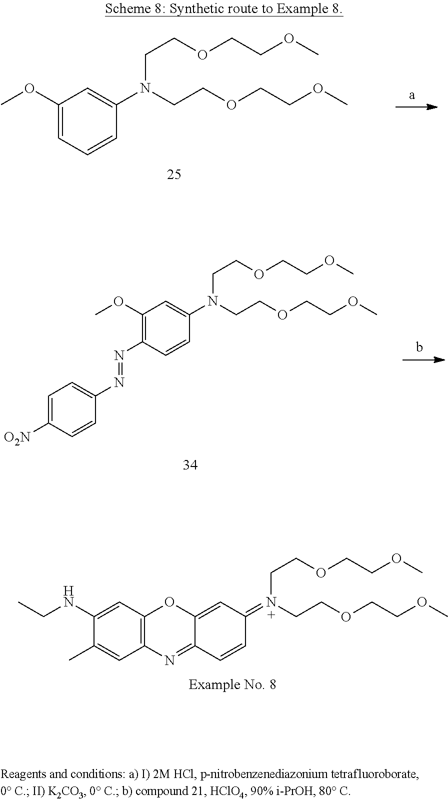

### EXAMPLE NO. 8

### N-(7-(ethylamino)-8-methyl-3H-phenoxazin-3-ylidene)-2-(2-methoxyethoxy)-N-(2-(2-methoxyethoxy)ethyl)ethan-1-aminium

(E)-3-methoxy-N,N-bis(2-(2-methoxyethoxy)ethyl)-4-((4-nitrophenyl)diazenyl)aniline (34): Compound 25 (246 mg, 0.751 mmol) was dissolved in MeOH (1 mL). The solution was chilled in an ice bath, then was treated with HCl (2 M, 10 mL). After 15 mins, p-nitrobenzenediazonium tetrafluoroborate (187 mg, 0.789 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

N-(7-(ethylamino)-8-methyl-3H-phenoxazin-3-ylidene)-2-(2-methoxyethoxy)-N-(2-(2-methoxyethoxy)ethyl)ethan-1-aminium (Example 8): Compound 21 (50 mg, 0.304 mmol) was dissolved in a solution of i-PrOH/H₂O (9:1, 5 mL) at 80° C. for 30 min. Compound 34 (145 mg, 0.304 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 8 (54 mg, 39%).

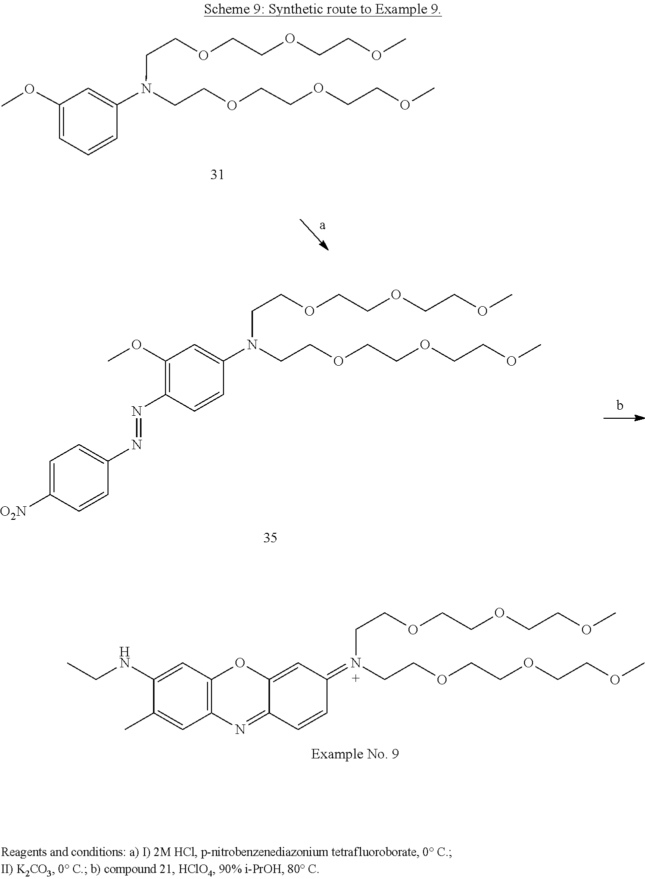

### EXAMPLE NO. 9

### N-(7-(ethylamino)-8-methyl-3H-phenoxazin-3-ylidene)-2-(2-(2-methoxyethoxy)ethoxy)-N-(2-(2-(2-methoxyethoxy)ethoxy)ethyl)ethan-1-aminium

(E)-3-methoxy-N,N-bis(2-(2-(2-methoxyethoxy)ethoxy)ethyl)-4-((4-nitrophenyl)diazenyl)aniline (35): Compound 31 (300 mg, 0.722 mmol) was dissolved in MeOH (1 mL). The solution was chilled in an ice bath, then was treated with HCl (2 M, 10 mL). After 15 mins, p-nitrobenzenediazonium tetrafluoroborate (180 mg, 0.758 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

N-(7-(ethylamino)-8-methyl-3H-phenoxazin-3-ylidene)-2-(2-(2-methoxyethoxy)ethoxy)-N-(2-(2-(2-methoxyethoxy)ethoxy)ethyl)ethan-1-aminium (Example 9): Compound 21 (30 mg, 0.183 mmol) was dissolved in a solution of i-PrOH/H₂O (9:1, 4 mL) at 80° C. for 30 min. Compound 35 (103 mg, 0.183 mmol) was added to the solution above in 3 portions over 15 mins. The reaction mixture was then treated with HClO₄ (70%, 40 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 9 (27 mg, 27%).

### EXAMPLE NO. 10

### (E)-N-ethyl-8-methyl-3-(2,5,11,14,17-pentaoxa-8λ⁴-azaoctadecan-8-ylidene)-3H-phenoxazin-7-amine

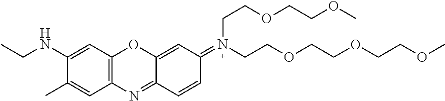

In addition to tosylate compounds 23 (synthesis of Example No. 6) and 29 (synthesis of Example No. 7), above, it is understood that compounds of the present disclosure may be made with the use of additional tosylate compounds known in the art. Illustrative and non-limiting examples include:
2-methoxyethyl 4-methylbenzenesulfonate (CAS Reg. No. 17178-10-8);
2-ethoxyethyl 4-methylbenzenesulfonate (CAS Reg. No. 17178-11-9);
2-(vinyloxy)ethyl 4-methylbenzenesulfonate (CAS Reg. No. 99051-18-0);
2-propoxyethyl 4-methylbenzenesulfonate (CAS Reg. No. 52497-47-9);
2-isopropoxyethyl 4-methylbenzenesulfonate (CAS Reg. No. 51218-98-5);
2-(allyloxy)ethyl 4-methylbenzenesulfonate (CAS Reg. No. 50563-72-9);
2-isobutoxyethyl 4-methylbenzenesulfonate (CAS Reg. No. 1852889-86-1);
2-(tert-butoxy)ethyl 4-methylbenzenesulfonate (CAS Reg. No. 108366-80-9);
2-((2-methylallyl)oxy)ethyl 4-methylbenzenesulfonate (CAS Reg. No. 64011-00-3);
2-(but-3-en-2-yloxy)ethyl 4-methylbenzenesulfonate (CAS Reg. No. 1628446-55-8);
2-(isopentyloxy)ethyl 4-methylbenzenesulfonate (CAS Reg. No. 915184-71-3);
2-(but-3-yn-1-yloxy)ethyl 4-methylbenzenesulfonate (CAS Reg. No. 1418561-91-7);
2-(2-methoxyethoxy)ethyl 4-methylbenzenesulfonate (CAS Reg. No. 50586-80-6);
2-(2-ethoxyethoxy)ethyl 4-methylbenzenesulfonate (CAS Reg. No. 54176-27-1);
2-(2-(vinyloxy)ethoxy)ethyl 4-methylbenzenesulfonate (CAS Reg. No. 117731-86-9);
2-(2-propoxyethoxy)ethyl 4-methylbenzenesulfonate (CAS Reg. No. 1709852-20-9);
2-(2-(allyloxy)ethoxy)ethyl 4-methylbenzenesulfonate (CAS Reg. No. 84183-96-0);
2-(2-(pentyloxy)ethoxy)ethyl 4-methylbenzenesulfonate (CAS Reg. No. 50964-16-4);
2-(2-(prop-2-yn-1-yloxy)ethoxy)ethyl 4-methylbenzenesulfonate (CASE Reg. No. 1119249-30-7);
2-(2-(tert-butoxy)ethoxy)ethyl 4-methylbenzenesulfonate (CAS Reg. No. 1431853-87-0);
2-(2-(isopentyloxy)ethoxy)ethyl 4-methylbenzenesulfonate (CAS Reg. No. 1359296-24-4);
2-(2-(pentyloxy)ethoxy)ethyl 4-methylbenzenesulfonate (CAS Reg. No. 2248492-16-0);
2-(2-(hexyloxy)ethoxy)ethyl 4-methylbenzenesulfonate (CAS Reg. No. 187748-60-3);
2-(2-(2-methoxyethoxy)ethoxy)ethyl 4-methylbenzenesulfonate (CAS Reg. No. 62921-74-8);
2-(2-(2-ethoxyethoxy)ethoxy)ethyl 4-methylbenzenesulfonate (CAS Reg. No. 62921-75-9);
2-(2-(2-propoxyethoxy)ethoxy)ethyl 4-methylbenzenesulfonate (CAS Reg. No. 64820-20-8);
2-(2-(2-(allyloxy)ethoxy)ethoxy)ethyl 4-methylbenzenesulfonate (CAS Reg. No. 84183-97-1);
2-(2-(2-(prop-2-yn-1-yloxy)ethoxy)ethoxy)ethyl 4-methylbenzenesulfonate (CAS Reg. No. 888009-94-7);
11-ethoxy-3,6,9,12-tetraoxatetradecyl 4-methylbenzenesulfonate (CAS Reg. No. 881920-24-7);
14-ethoxy-3,6,9,12,15-pentaoxaheptadecyl 4-methylbenzenesulfonate (CAS Reg. No. 1630091-41-6);
2,5,9,12-tetraoxatridecan-7-yl 4-methylbenzenesulfonate (CAS Reg. No. 1644403-15-5);
2,5,8,12,15,18-hexaoxanonadecan-10-yl 4-methylbenzenesulfonate (CAS Reg. No. 1644403-20-2);
3,6,9,13,16,19-hexaoxahenicosan-11-yl 4-methylbenzenesulfonate (CAS Reg. No. 508224-19-9);
2,5,8,11,15,18,21,24-octaoxapentacosan-13-yl 4-methylbenzenesulfonate (CAS Reg. No. 214851-23-7);
2,5,8,11,14,18,21,24,27,30-decaoxahentriacontan-16-yl 4-methylbenzenesulfonate (CAS Reg. No.
2346589-70-4);
2-(2-((tert-butyldimethylsilyl)oxy)ethoxy)ethyl 4-methylbenzenesulfonate (CAS Reg. No. 131326-40-4); and
2,2-dimethyl-3,6,9-trioxa-2-silaundecan-11-yl 4-methylbenzenesulfonate (CAS Reg. No. 472968-83-5).
2,2,3,3-tetramethyl-4,7,10-trioxa-3-siladodecan-12-yl 4-methylbenzenesulfonate (CAS Reg. No. 199484-66-7).

Methods of Use. The formulations of the disclosure can be used to image nerves or nerve tissue. In particular embodiments, the formulations of the disclosure can be used to image nerves or nerve tissue in a subject. In particular embodiments, images of nerves can be obtained intraoperatively during FGS. In particular embodiments, the visualization of nerves during FGS allows surgery to be performed on tissue of interest while sparing nerves so as to reduce incidence of nerve injury during surgery. The area where surgery is performed or nearby regions can be surgically exposed. Surgery can be performed on organs, which include tissues such as nerve tissue, muscle tissue, and adipose tissue. The surgery can be laparoscopic, which is minimally invasive and includes the use of a thin, tubular device (laparoscope) that is inserted through a keyhole incision into a part of a subject's body, such as the abdomen or pelvis. The surgery can be assisted by a robot. Robot-assisted surgery can offer more precision, flexibility, and control, and is often associated with minimally invasive surgery.

A subject refers to any animal. The animal may be a mammal. Examples of suitable mammals include human and non-human primates, dogs, cats, sheep, cows, pigs, horses, mice, rats, rabbits, and guinea pigs.

A formulation of the disclosure can be directly applied to a tissue for imaging of nerves. In particular embodiments, direct application includes applying the formulation topically to a tissue to be imaged. In particular embodiments, direct application includes any route of application characterized by physical breaching of a tissue of a subject and application of the composition through the breach in the tissue. Direct application of a formulation includes application of a formulation to a tissue by injection, through a surgical incision, through a tissue-penetrating non-surgical wound, and the like. In particular embodiments, the tissue is undergoing surgery. In particular embodiments, direct application of a formulation includes applying the formulation to a surgical area, to nerves or nerve tissue, and to an exposed nerve. In particular embodiments, direct application of a formulation includes subcutaneous, intraperitoneal, or intramuscular application. In particular embodiments, a formulation can be delivered through a syringe or tubing to tissue. In particular embodiments, the tissue is ex vivo and is not undergoing surgery.

In particular embodiments, the fluorophore concentration in a formulation that is directly applied to nerve tissue includes a concentration range of 40 to 300 μg/mL. In particular embodiments, the fluorophore concentration in a formulation for direct application includes 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, and 200 μg/mL. In particular embodiments, the fluorophore concentration in a formulation for direct application is 50 μg/mL In particular embodiments, the fluorophore concentration in a formulation for direct application is 200 μg/mL.

A formulation of the disclosure can be systemically applied to a subject for imaging of nerves. In particular embodiments, systemic application of a formulation includes intravenous injection of the formulation into a subject.

A formulation that is directly applied to a tissue can be allowed to penetrate the tissue for a given amount of time after direct application. In particular embodiments, the formulation can be allowed to penetrate the tissue for 30 seconds to 15 minutes, for 1 to 10 minutes, for 1 to 5 minutes, for 1 minute, for 2 minutes, for 3 minutes, for 4 minutes, or for 5 minutes. In particular embodiments, the formulation can be allowed to penetrate the tissue for 1 to 2 minutes. A formulation that is systemically applied to a subject can be administered a sufficient time before imaging such that the formulation can reach the area to be imaged and is present in such area at the time of imaging. In particular embodiments, a formulation that is systemically applied to a subject can be administered a sufficient time prior to imaging to allow uptake of the formulation by tissue in the subject. In particular embodiments, the formulation may be administered up to or less than 30 minutes, 1 hour, 2 hours, 3 hours, 4 hours, 5 hours, 6 hours, 7 hours, or 8 hours before imaging. The amount of time required may depend on the nerve imaging application and the administration site. In particular embodiments, the formulation is administered no more than 30 minutes, 1 hour, 2 hours, 3 hours, or 4 hours before imaging. In particular embodiments, the formulation is administered no more than 2 hours before imaging.

Tissue stained by a formulation including a fluorophore by direct application can be washed with buffer prior to imaging of the stained tissue. Washing of tissue stained by a formulation including a fluorophore can include flushing the tissue with an appropriate buffer and removing the buffer. In particular embodiments, the stained tissue can be washed 1 to 18 times, 1 to 10 times, 1 to 6 times, 1 time, 2 times, 3 times, 4 times, 5 times, or 6 times, with wash buffer. In particular embodiments, the stained tissue can be washed 6 times. In particular embodiments, the wash buffer is phosphate-buffered saline (PBS). In particular embodiments, washing the stained tissue removes unbound fluorophore. In particular embodiments, washing the stained tissue increases the nerve signal intensity and/or the signal to background ratio (SBR) as compared to no washing of the stained tissue. In particular embodiments, washing the stained tissue resolubilizes the flurorophore and allows for further diffusion of the fluorophore into the nerve tissue.

Imaging a tissue stained by a formulation including a fluorophore includes applying light to tissue that has been stained with a formulation of the disclosure. The light can be at a wavelength sufficient to excite the fluorophore in the formulation to fluoresce. In particular embodiments, light to excite the fluorophore is at a wavelength in the near infrared spectra. In particular embodiments, the fluorophore of a formulation emits at a wavelength in the near infrared spectra. In particular embodiments, the near infrared spectra includes a wavelength of 700 to 900 nm.

Imaging a tissue stained by a formulation including a fluorophore includes obtaining fluorescence images of the stained tissue by optical imaging systems such as ones described in the Examples.

In particular embodiments, imaging a tissue includes observing fluorescence images of the stained tissue. The fluorescence images can include still images (whether printed or on screen), or real-time images on a video monitor. In particular embodiments, the individual images of nerves obtained by staining of the nerves with the present formulations can be used for diagnostic purposes and for documentation of nerve location. By observing the fluorescence images the surgical team can determine the absence or presence of a nerve in the image. The surgical team can thus use information about the presence/absence or location of one or more nerves to determine how they will perform the surgical procedure. For example, based on information obtained through the disclosed methods, the surgical team may decide to perform a surgical cut at a point in the tissue where they are less likely to inadvertently cut or surgically contact a particular nerve based on the perceived absence of a nerve in an area of the tissue.

The information obtained from the obtained image can aid in grafting the ends of the nerves if they are transected. In the event of transection, nerve grafts can be applied directly to the ends to facilitate sprouting of regenerative neural fibers. In this case, the light visible from the fluorescence of the ends of transected nerves provides a target to guide the anastomosis of the nerves by the nerve graft.

Kits. Formulations of the present disclosure to detect nerve tissue can also be provided as kits. Kits for detecting nerve tissue can include: a gel-based formulation including: (i) a fluorophore, and (ii) 5-10% sodium alginate and/or 18-26% PEO-PPO-PEO triblock copolymer; a formulation including (i) a fluorophore, and (ii) a DSPE-PEG micelle and/or cyclodextrin; and/or wash buffers. Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding: directly applying the formulations to a tissue; washing to remove excess formulation; systemically administering the formulations to a subject; applying light for visualization of the fluorophores; capturing fluorescent images of the tissue; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary laboratory and/or medical supplies needed to use the kit effectively, such as syringes, ampules, tubing, gloves, tubes, buffers, and the like. Variations in contents of any of the kits described herein can be made.

### EXEMPLARY EMBODIMENTS

1. A gel-based formulation for tissue imaging including (i) a fluorophore, and (ii) 5-10% alginate salt and/or 18-26% PEO-PPO-PEO triblock copolymer.
2. The gel-based formulation of embodiment 1 wherein the fluorophore is an oxazine derivative.
3. The gel-based formulation of embodiment 2 wherein the oxazine derivative is LGW01-08.
4. The gel-based formulation of any one of embodiments 1-3 including 50 μg/mL fluorophore.
5. The gel-based formulation of any one of embodiments 1-3 including 200 μg/mL fluorophore.
6. The gel-based formulation of any one of embodiments 1-5 wherein the alginate salt is sodium alginate.
7. The gel-based formulation of embodiment 6 including 5-9% sodium alginate.
8. The gel-based formulation of embodiment 6 or 7 including 5-8% sodium alginate.
9. The gel-based formulation of any one of embodiments 6-8 including 6-8% sodium alginate.
10. The gel-based formulation of any one of embodiments 6-9 including 6-7% sodium alginate.
11. The gel-based formulation of any one of embodiments 6-10 including 6.5% sodium alginate.
12. The gel-based formulation of any one of embodiments 1-11 including 19-25% PEO-PPO-PEO triblock copolymer.
13. The gel-based formulation of any one of embodiments 1-12 including 20-24% PEO-PPO-PEO triblock copolymer.
14. The gel-based formulation of any one of embodiments 1-13 including 21-23% PEO-PPO-PEO triblock copolymer.
15. The gel-based formulation of any one of embodiments 1-14 including 22% PEO-PPO-PEO triblock copolymer.
16. The gel-based formulation of any one of embodiments 6-13 including 5-8% sodium alginate and/or 20-24% PEO-PPO-PEO block copolymer.
17. The gel-based formulation of any one of embodiments 10-13 including 6-7% sodium alginate and/or 20-24% PEO-PPO-PEO block copolymer.
18. The gel-based formulation of any one of embodiments 10-14 including 6-7% sodium alginate and/or 21-23% PEO-PPO-PEO block copolymer.
19. A method of directly applying the gel-based formulation of any one of embodiments 1-18 including applying the gel-based formulation to an exposed nerve.
20. The method of embodiment 19 wherein the applying is during radical prostatectomy.
21. The method of embodiment 19 or 20 further including washing the applied gel-based formulation from the nerve.
22. The method of any one of embodiments 19-21 wherein the washing includes 5-7 flushes.
23. A formulation for systemic administration tissue imaging including (i) a fluorophore, and (ii) a DSPE-PEG micelle and/or cyclodextrin.
24. The formulation of embodiment 23 wherein the fluorophore is an oxazine derivative.
25. The formulation of embodiment 24 wherein the oxazine derivative is LGW01-08.
26. The formulation of any one of embodiments 23-25 including a DSPE-PEG micelle with the fluorophore encapsulated at 0.5-0.9 mg/mL.
27. The formulation of any one of embodiments 23-26 including a DSPE-PEG micelle with the fluorophore encapsulated at 0.7 mg/mL.
28. The formulation of any one of embodiments 23-25 including cyclodextrin with the fluorophore encapsulated at 0.5-1.2 mg/mL.
29. The formulation of any one of embodiments 23-25 or 28 including cyclodextrin with the fluorophore encapsulated at 0.7-1.0 mg/mL.
30. A method of staining a nerve or tissue including systemically administering a formulation of any of embodiments 23-29 to a subject during an operative procedure.
31. The method of embodiment 30 wherein the administering is at dose of 2.5 mg/kg.
32. A method of detecting nerves intraoperatively in a subject undergoing surgery including:
   directly applying a gel-based formulation including (i) a fluorophore, and (ii) 5-10% alginate salt and/or 18-26% PEO-PPO-PEO triblock copolymer to stain tissue undergoing surgery; and
   imaging the stained tissue, thereby detecting nerves intraoperatively in the subject undergoing surgery.
33. The method of embodiment 32, further including washing the tissue with buffer after applying the gel-based formulation and prior to imaging the stained tissue.
34. The method of embodiment 33, wherein the washing removes unbound fluorophore.
35. The method of embodiment 33 or 34, wherein the buffer is phosphate-buffered saline (PBS).
36. The method of any one of embodiments 33-35, further including allowing the gel-based formulation to penetrate the tissue for 30 seconds to 5 minutes prior to the washing.
37. The method of any one of embodiments 33-36, further including allowing the gel-based formulation to penetrate the tissue for 1 minute to 2 minutes prior to the washing.
38. The method of any one of embodiments 32-37, wherein risk of iatrogenic injury to the subject undergoing surgery is reduced.
39. The method of any one of embodiments 32-38, wherein the surgery is laparoscopic.
40. The method of any one of embodiments 32-39, wherein the surgery is performed by a robot.
41. The method of any one of embodiments 32-40, wherein the surgery is radical prostatectomy.
42. The method of any one of embodiments 32-41, wherein the fluorophore is an oxazine derivative.
43. The method of any one of embodiments 32-42, wherein the oxazine derivative is LGW01-08.
44. The method of any one of embodiments 32-43, wherein the concentration of the fluorophore is 50 μg/mL
45. The method of any one of embodiments 32-43, wherein the concentration of the fluorophore is 200 μg/mL
46. The method of any one of embodiments 32-45 wherein the alginate salt is sodium alginate.
47. The method of embodiment 46 including 5-9% sodium alginate.
48. The method of embodiment 46 or 47 including 5-8% sodium alginate.
49. The method of any one of embodiments 46-48 including 6-8% sodium alginate.
50. The method of any one of embodiments 46-49 including 6-7% sodium alginate.
51. The method of any one of embodiments 46-50 including 6.5% sodium alginate.
52. The method of any one of embodiments 46-51 including 19-25% PEO-PPO-PEO triblock copolymer.
53. The method of any one of embodiments 46-52 including 20-24% PEO-PPO-PEO triblock copolymer.
54. The method of any one of embodiments 46-53 including 21-23% PEO-PPO-PEO triblock copolymer.
55. The method of any one of embodiments 46-54 including 22% PEO-PPO-PEO triblock copolymer.
56. The method of any one of embodiments 46-53 including 5-8% sodium alginate and/or 20-24% PEO-PPO-PEO block copolymer.
57. The method of any one of embodiments 50-53 including 6-7% sodium alginate and/or 20-24% PEO-PPO-PEO block copolymer.
58. The method of any one of embodiments 50-54 including 6-7% sodium alginate and/or 21-23% PEO-PPO-PEO block copolymer.
59. A method of detecting nerves within ex vivo tissue including:
   directly applying a gel-based formulation including (i) a fluorophore, and (ii) 5-10% alginate salt and/or 18-26% PEO-PPO-PEO triblock copolymer to stain the ex vivo tissue; and
   imaging the stained ex vivo tissue, thereby detecting nerves within the ex vivo tissue.
60. The method of embodiment 59, further including washing the ex vivo tissue with buffer after applying the gel-based formulation and prior to imaging the stained ex vivo tissue.
61. The method of embodiment 59 or 60, wherein the washing removes unbound fluorophore.
62. The method of embodiment 60 or 61, wherein the buffer is phosphate-buffered saline (PBS).
63. The method of any one of embodiments 60-62, further including allowing the gel-based formulation to penetrate the tissue for 30 seconds to 5 minutes prior to the washing.
64. The method of any one of embodiments 60-63, further including allowing the gel-based formulation to penetrate the tissue for 1 minute to 2 minutes prior to the washing.
65. The method of any one of embodiments 59-64, wherein the fluorophore is an oxazine derivative.
66. The method of any one of embodiments 59-65, wherein the oxazine derivative is LGW01-08
67. The method of any one of embodiments 59-66, wherein the concentration of the fluorophore is 50 μg/mL
68. The method of any one of embodiments 59-66, wherein the concentration of the fluorophore is 200 μg/mL
69. The method of any one of embodiments 59-68 wherein the alginate salt is sodium alginate.
70. The method of embodiment 69 including 5-9% sodium alginate.
71. The method of embodiment 69 or 70 including 5-8% sodium alginate.
72. The method of any one of embodiments 69-71 including 6-8% sodium alginate.
73. The method of any one of embodiments 69-72 including 6-7% sodium alginate.
74. The method of any one of embodiments 69-73 including 6.5% sodium alginate.
75. The method of any one of embodiments 69-74 including 19-25% PEO-PPO-PEO triblock copolymer.
76. The method of any one of embodiments 69-75 including 20-24% PEO-PPO-PEO triblock copolymer.
77. The method of any one of embodiments 69-76 including 21-23% PEO-PPO-PEO triblock copolymer.
78. The method of any one of embodiments 69-77 including 22% PEO-PPO-PEO triblock copolymer.
79. The method of any one of embodiments 69-76 including 5-8% sodium alginate and/or 20-24% PEO-PPO-PEO block copolymer.
80. The method of any one of embodiments 73-76 including 6-7% sodium alginate and/or 20-24% PEO-PPO-PEO block copolymer.
81. The method of any one of embodiments 73-77 including 6-7% sodium alginate and/or 21-23% PEO-PPO-PEO block copolymer.
82. A method of detecting nerves intraoperatively in a subject undergoing surgery including: systemically administering a formulation (i) a fluorophore, and (ii) a DSPE-PEG micelle and/or cyclodextrin to the subject before or during surgery; and imaging stained tissue undergoing surgery in the subject, thereby detecting nerves intraoperatively in the subject undergoing surgery.
83. The method of embodiment 82, wherein systemically administering includes intravenously injecting the subject with the formulation.
84. The method of embodiment 82 or 83, including systemically administering the formulation 30 minutes to 4 hours prior to the imaging.
85. The method of embodiment 82 or 83, including systemically administering the formulation 2 hours prior to the imaging.
86. The method of any one of embodiments 82-85, wherein risk of iatrogenic injury to the subject undergoing surgery is reduced.
87. The method of any one of embodiments 82-86, wherein the surgery is laparoscopic.
88. The method of any one of embodiments 82-87, wherein the surgery is performed by a robot.
89. The method of any one of embodiments 82-88, wherein the fluorophore is an oxazine derivative.
90. The method of any one of embodiments 82-89, wherein the oxazine derivative is LGW01-08.
91. The method of any one of embodiments 82-90 including a DSPE-PEG micelle with the fluorophore encapsulated at 0.5-0.9 mg/mL.
92. The method of any one of embodiments 82-91 including a DSPE-PEG micelle with the fluorophore encapsulated at 0.7 mg/mL.
93. The method of any one of embodiments 82-90 including cyclodextrin with the fluorophore encapsulated at 0.5-1.2 mg/mL.
94. The method of any one of embodiments 82-90 or 93 including cyclodextrin with the fluorophore encapsulated at 0.7-1.0 mg/mL.
95. A kit including:
   (a) a gel-based formulation including (i) a fluorophore, and (ii) 5-10% alginate salt and/or 18-26% PEO-PPO-PEO triblock copolymer; and/or
   (b) a formulation including (i) a fluorophore, and (ii) a DSPE-PEG micelle and/or cyclodextrin; and
   (c) use instructions for applying the formulation of (a) and/or administering the formulation of (b).
96. The kit of embodiment 95, wherein the fluorophore is an oxazine derivative.
97. The kit of embodiment 96, wherein the oxazine derivative is LGW01-08.
98. The kit of any one of embodiments 95-97, wherein the concentration of the fluorophore is 50 μg/mL.
99. The kit of any one of embodiments 95-97, wherein the concentration of the fluorophore is 200 μg/mL.
100. The kit of any one of embodiments 95-99 wherein the alginate salt is sodium alginate.
101. The kit of embodiment 100 including 5-9% sodium alginate.
102. The kit of embodiment 100 or 101 including 5-8% sodium alginate.
103. The kit of any one of embodiments 100-102 including 6-8% sodium alginate.
104. The kit of any one of embodiments 100-103 including 6-7% sodium alginate.
105. The kit of any one of embodiments 100-104 including 6.5% sodium alginate.
106. The kit of any one of embodiments 95-105 including 19-25% PEO-PPO-PEO triblock copolymer.
107. The kit of any one of embodiments 95-106 including 20-24% PEO-PPO-PEO triblock copolymer.
108. The kit of any one of embodiments 95-107 including 21-23% PEO-PPO-PEO triblock copolymer.
109. The kit of any one of embodiments 95-108 including 22% PEO-PPO-PEO triblock copolymer.
110. The kit of any one of embodiments 100-107 including 5-8% sodium alginate and/or 20-24% PEO-PPO-PEO block copolymer.
111. The kit of any one of embodiments 104-107 including 6-7% sodium alginate and/or 20-24% PEO-PPO-PEO block copolymer.
112. The kit of any one of embodiments 104-108 including 6-7% sodium alginate and/or 21-23% PEO-PPO-PEO block copolymer.
113. The kit of any one of embodiments 95-112 including a DSPE-PEG micelle with the fluorophore encapsulated at 0.5-0.9 mg/mL.
114. The kit of any one of embodiments 95-113 including a DSPE-PEG micelle with the fluorophore encapsulated at 0.7 mg/mL.
115. The kit of any one of embodiments 95-112 including cyclodextrin with the fluorophore encapsulated at 0.5-1.2 mg/mL.
116. The kit of any one of embodiments 95-112 or 115 including cyclodextrin with the fluorophore encapsulated at 0.7-1.0 mg/mL.

### EXAMPLE 1

### Improved Formulations for Direct Administration of Nerve Specific Probes for Fluorescence Image Guided Surgery

This example provides clinically viable formulations of a novel near-infrared nerve specific oxazine fluorophore, LGW01-08, that possess unique gelling characteristics. The formulations are useful for direct (local) administration to a subject, allowing an excellent platform for clinical translation of nerve-specific fluorophores for fluorescence guided surgery (FGS).

Iatrogenic nerve injury significantly affects surgical outcomes for procedures like the radical prostatectomy (RP), with up to 60% of patients reporting nerve damage one to two years post-surgery. Although nerve sparing RP techniques have been practices for over 30 years, it remains difficult for surgeons to identify nerve tissue intraoperatively and nerve sparing success rates are strongly correlated with experience level. Fluorescence guided surgery (FGS) offers a potential solution for improved nerve sparing by providing direct visualization of nerve tissue intraoperatively. However, novel probes for FGS face an extraordinary regulatory challenge to achieve clinical translation. Previously, a direct administration methodology was developed that enabled application of nerve-specific fluorophores at a much lower dose than systemic administration for clinical translation via exploratory IND guidance. However, a clinically viable formulation was necessary to advance this promising technology to clinical use. Previously a non-FDA approved co-solvent formulation was utilized which resulted in significant background staining in preliminary large animal studies from a lack of staining control inherent to liquid-based formulations. In the present study, we report on the development of a clinically viable gel-based formulation strategy that enables direct administration of a nerve-specific fluorescent contrast agent with increased control for nerve sparing FGS applications. An F127 Pluronic® formulation is used to solubilize the novel near-infrared nerve specific oxazine fluorophores, providing increased staining control for a variety of tissue surfaces, angles, and morphologies. Additionally, the formulation developed herein possesses unique gelling characteristics, allowing it to easily be spread as a liquid followed by rapid gelling at body temperature for subsequent tissue hold. Further optimization of the direct administration protocol has decreased the total staining time to 1-2 minutes, improving compatibility with surgical procedures. The resulting gel formulation and direct administration methodology provides an excellent platform for clinical translation of novel nerve-specific fluorophores for FGS.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a significant decrease in SBR at a site of surgical intervention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

### Definitions

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

A “subject” or a “patient” refers to any animal. The animal may be a mammal. Examples of suitable mammals include human and non-human primates, dogs, cats, sheep, cows, pigs, horses, mice, rats, rabbits, and guinea pigs. In some embodiments the subject or patient is a human, particularly including a human undergoing or in need of a surgical procedure or examination.

The term “nerve” used herein means a bundle of neural axons. Within a nerve, each axon is surrounded by a layer of connective tissue called the endoneurium. The axons are bundled together into groups called fascicles, and each fascicle is wrapped in a layer of connective tissue called the perineurium. The entire nerve is wrapped in a layer of connective tissue called the epineurium. The term “nerve” is intended to include any tissues (e.g., the sinoatrial node or the atriventricular node) or structures associated therewith (e.g., neuromuscular junctions).

The term “nerve-specific” or “nerve specific” herein refers to an agent that is drawn to a nerve or nerve tissue and may be used in fluorescent imaging techniques to help contrast and differentiate the nerve or nerve tissue from surrounding cells and/or tissues. The term “nerve specificity” refers to the nature or activity of an agent being nerve-specific.

The term “near infrared” or the acronym “(NIR)” refers to light at the near infrared spectrum, generally at a wavelength of about 0.65 to about 1.4 μm (700 nm-1400 nm. It may also refer to a range designated by the International Organization for Standardization as from a wavelength of about 0.78 μm to about 3 μm. In some embodiments, the preferred near infrared spectroscopy and imaging (NIRS) range is from about 650 nm to about 950 nm. In other embodiments, the preferred near infrared spectroscopy and imaging (NIRS) range is from about 650 nm to about 900 nm.

In some embodiments the agents and/or compositions comprising them are intended for direct/topical administration. Direct or topical administration are understood herein to comprise the administration of an agent or composition directly to surface of a tissue, organ, nerve bundle, or other bodily component. In some methods, the administration may be accomplished by brushing, spraying, or irrigation with the appropriate compound or composition.

In other embodiments, the agents and/or compositions may be administered systemically to the patient or subject, such as through intravenous injection or infusion.

In other embodiments, the agents and/or compositions may be administered locally to a desired tissue or organ, such as through injection.

The terms “effective amount” or “medically effective amount” or “imaging effective amount”, or like terms refers to an amount of a compound or composition as described herein to cover a target area sufficiently to complete binding to one or more nerves such that they may be identified through relevant imaging techniques, particularly near-infrared imaging techniques.

The term “label” refers to a molecule that facilitates the visualization and/or detection of a targeting molecule disclosed herein. In some embodiments, the label is a fluorescent moiety. The term “labeling” refers to a successful administration of the label to a target to allow such detection.

As used herein, the terms “robotic surgery”, “robot-assisted surgery”, or “computer-assisted surgery” refer to surgical techniques involving robotic systems that control the movement of medical instruments to conduct a surgical procedure with precise, flexible, and/or minimally invasive actions designed to limit the amount of surgical trauma, blood loss, pain, scarring, and post-surgical patient recovery time and/or complications, such as infection at the surgical area. Examples of robotic surgery include those conducted using the da Vinci Surgical System (Intuitive Surgical, Sunnyvale, Calif., USA) approved by the U.S. Food and Drug Administration in 2000.

The terms “surgery” or “surgical method” as used herein, refers to any method used to manipulate, change, or cause an effect by a physical intervention. These methods include, but are not limited to open surgery, endoscopic surgery, laparoscopic surgery, minimally invasive surgery, robotic surgery, any procedures that may affect any neuron or nerve, such as placement of retractors during spinal surgery, electrically conducting cardiac tissue or nerve ablation, epidural injection, intrathecal injections, neuron or nerve blocks, implantation of devices such as neuron or nerve stimulators and implantation of pumps. These methods may also include biopsy or other invasive techniques for the collection of cell or tissue samples, such as for diagnostic purposes.

As used herein, the term “targeting molecule” refers to any agent (e.g., peptide, protein, nucleic acid polymer, aptamer, or small molecule) that associates with (e.g., binds to) a target of interest. The target of interest may be a nerve cell or an organ or tissue associated with one or more nerve cells or nerve structures. In some embodiments, the targeting molecule is any agent that associates with (e.g., binds to) a target comprising one or more neurons, nerves, or tissues or structures associated therewith, i.e. nerve tissues, nervous system tissues, nerve bundles, etc. It is understood that nerve and nerve-related targets include those associated with the brain and spinal cord of the central nervous system (CNS) and the nerves of the peripheral nervous system (PNS).

The term “prostatectomy” refers to a surgical technique to remove all or part of a subject's prostate gland. A “radical prostatectomy” concerns removal of a subject's entire prostate gland, along with surrounding tissues, often including the seminal vesicles and nearby lymph nodes.

The terms “orthopedic limb repair” or “orthopedic limb repair surgeries” refer to surgical techniques performed on the limb musculoskeletal system of a subject. These techniques include limb reconstruction surgeries, joint replacement procedures, revision joint surgery, debridement, bone fusions, tendon or ligament repair, internal fixation of bone, and osteotamies.

The term “fluorophore” herein refers to any one of the compounds described herein for use in imaging techniques, particularly for nerve imaging techniques. Each of the compounds described herein as the product of a specific synthesis or described in a generic description is considered a fluorophore for methods, uses, and compositions.

The term “variable” or “variables” used in the generic descriptions and claims herein refer to the entities or moieties that may, in some instances, be chosen from a specified group. Such variables may include R₁, R₂, R₃, R₄, R₅, R₆, n1, n2, n3, n4, X₁, and the like.

All ranges disclosed and/or claimed herein are inclusive of the recited endpoint and independently combinable (for example, the ranges of “from 2 to 10” and “2-10” are inclusive of the endpoints, 2 and 10, and all the intermediate values). For example, a reference to “Claims 2-5” includes all claims 2, 3, 4, and 5.

The term “intraoperatively” as used in describing methods or uses herein refers to an activity that occurs during a surgical procedure or in immediate preparation for such procedure.

As used herein, terms “pharmaceutically acceptable” or “physiologically acceptable”, and the like, used in regard to a formulation or composition component refer to a pharmaceutically acceptable vehicle that includes, without limitation, any and all carriers, solvents, dispersion media, coatings, antibacterial and antifungal agents, isotonic and absorption delaying agents and the like. Such materials provide an acceptable level of activity of the fluorophore/compound in question and are compatible with and substantially non-toxic to the cells, tissues, organs, etc. with which they contact. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

What is claimed is:

1. A gel-based formulation for tissue imaging comprising:
(i) 5-10% sodium alginate and/or 18-26% PEO-PPO-PEO triblock copolymer; and
(ii) an effective amount of a fluorophore compound of Formula (I): 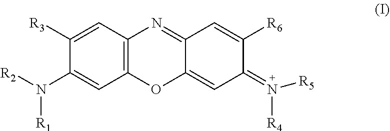
wherein:
R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 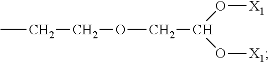
b) 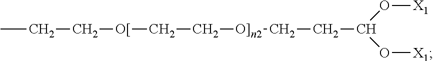
c) 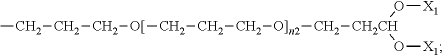
and
d) 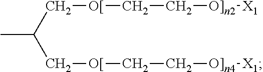
R₃ is hydrogen or R₂ and R₃ together form a fused ring, creating a core of Formula (II): 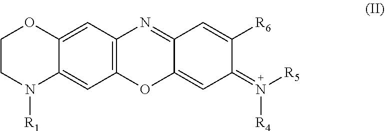
R₄ and R₅, together with the nitrogen atom to which they are bound, form a ring selected from the group of: 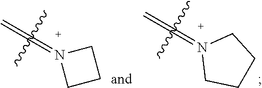
or, when the compound is of Formula (II), R₄ and R₅ may be independently selected from C₁-C₆ alkyl, with the proviso that, when R₄ is ethyl, R₅ is not ethyl;
R₆ is hydrogen;
or, when R₂ and R₃ together form a fused ring to create a core of Formula (II), R₅ and R₆ may also, together with the nitrogen atom to which R₅ is bound, form a fused ring, creating a core of Formula (III): 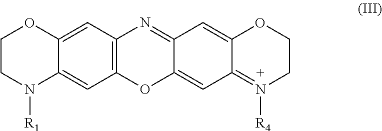
with the proviso that, when R₄ and R₅, together with the nitrogen atom to which they are bound, form the ring 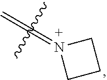
and R₃ is H, R₁ and R₂, together with the nitrogen atom to which they are bound, may form a pyrrolidinyl ring;
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n is an integer selected from the group of 1 and 2;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
with the proviso that the sum of n2+n2 is not greater than 10;
with the proviso that the sum of n2+n3 is not greater than 10;
with the proviso that the sum of n2+n4 is not greater than 10;
with the proviso that the sum of n3+n4 is not greater than 10;
with the proviso that, when R₄ and R₅, together with the nitrogen atom to which they are bound, form the ring 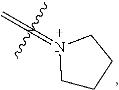
R₁ and R₂ are not both methyl, R₁ and R₂ are not both ethyl, R₁ and R₂ are not both n-propyl, R₁ and R₂ are not both n-butyl, and R₁ and R₂ are not both n-pentyl;
with the proviso that, when the compound is of Formula (III), when R₁ is methyl, R₄ is not methyl; and
with the proviso that, when the compound is of Formula (III), when R₁ is ethyl, R₄ is not ethyl.

2. The gel-based formulation for tissue imaging of claim 1, wherein the compound is of Formula (IV): 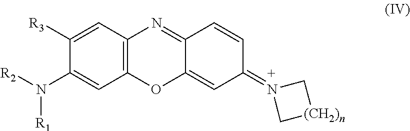
wherein:
R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
e) 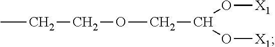
f) 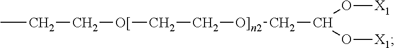
g) 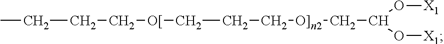
and
h) 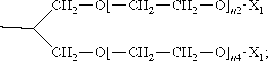
R₃ is hydrogen or R₂ and R₃ together form a fused ring, creating a core of Formula (V): 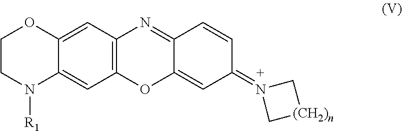
with the proviso that, when n is 1, and R₃ is H, R₁ and R₂, together with the nitrogen atom to which they are bound, may form a pyrrolidinyl ring;
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n is an integer selected from the group of 1 and 2;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
with the proviso that the sum of n2+n2 is not greater than 10;
with the proviso that the sum of n2+n3 is not greater than 10;
with the proviso that the sum of n2+n4 is not greater than 10;
with the proviso that the sum of n3+n4 is not greater than 10; and
with the proviso that, when n is 2, R₁ and R₂ are not both methyl, R₁ and R₂ are not both ethyl, R₁ and R₂ are not both n-propyl, R₁ and R₂ are not both n-butyl, and R₁ and R₂ are not both n-pentyl.

3. The gel-based formulation for tissue imaging of claim 1, wherein the compound is a compound of Formula (VI): 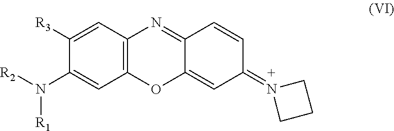
wherein:
R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 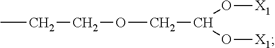
b) 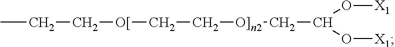
c) 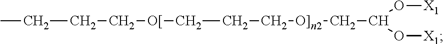
and
d) 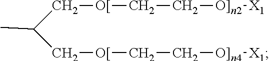
R₃ is hydrogen or R₂ and R₃ together form a fused ring, creating a core of Formula (V): 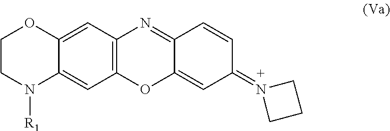
with the proviso that, when n is 1, and R₃ is H, R₁ and R₂, together with the nitrogen atom to which they are bound, may form a pyrrolidinyl ring;
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n is an integer selected from the group of 1 and 2;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
with the proviso that the sum of n2+n2 is not greater than 10;
with the proviso that the sum of n2+n3 is not greater than 10;
with the proviso that the sum of n2+n4 is not greater than 10; and
with the proviso that the sum of n3+n4 is not greater than 10.

4. The gel-based formulation for tissue imaging of claim 1, wherein the compound is a compound of Formula (VI): 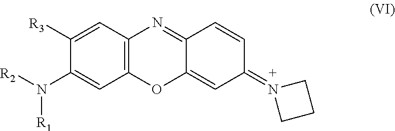
wherein R₃ is hydrogen, and R₁ and R₂ are selected independently from C₁-C₆ alkyl.

5. The gel-based formulation for tissue imaging of claim 4, wherein R₃ is hydrogen, and R₁ and R₂ are selected independently from methyl and ethyl.

6. The gel-based formulation for tissue imaging of claim 1, wherein the compound is a compound of Formula (VII): 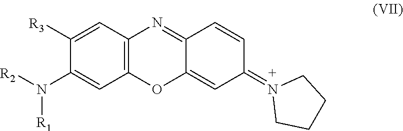
wherein:
R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 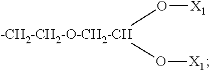
b) 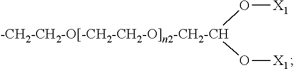
c) 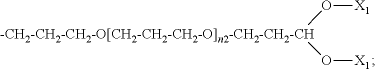
and
d) 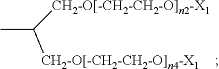
R₃ is hydrogen or R₂ and R₃ together form a fused ring, creating a core of Formula (V): 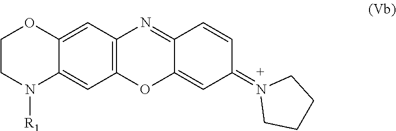
with the proviso that, when n is 1, and R₃ is H, R₁ and R₂, together with the nitrogen atom to which they are bound, may form a pyrrolidinyl ring;
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n is an integer selected from the group of 1 and 2;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
with the proviso that the sum of n2+n2 is not greater than 10;
with the proviso that the sum of n2+n3 is not greater than 10;
with the proviso that the sum of n2+n4 is not greater than 10;
with the proviso that the sum of n3+n4 is not greater than 10; and
with the proviso that R₁ and R₂ are not both methyl, the proviso that R₁ and R₂ are not both ethyl, the proviso that R₁ and R₂ are not both n-propyl, the proviso that R₁ and R₂ are not both n-butyl, and the proviso that R₁ and R₂ are not both n-pentyl.

7. The gel-based formulation for tissue imaging of claim 1, wherein the compound is a compound of Formula (VII): 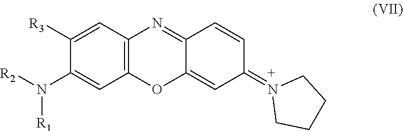
wherein R₃ is hydrogen, and R₁ and R₂ are selected independently from C₁-C₆ alkyl; with the proviso that, when R₁ is ethyl, R₂ is not ethyl.

8. The gel-based formulation for tissue imaging of claim 7, wherein R₃ is hydrogen, and R₁ and R₂ are selected independently from methyl and ethyl; with the proviso that, when R₁ is ethyl, R₂ is not ethyl.

9. The gel-based formulation for tissue imaging of claim 1, wherein the compound is a compound of the formula: 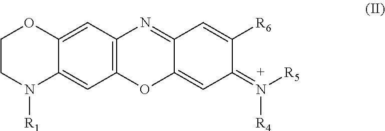
wherein:
R₁ is selected from the group of straight or branched C₁-C₆ alkyl;
—(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 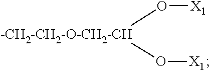
b) 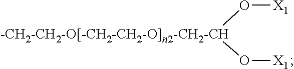
c) 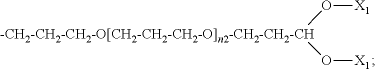
and 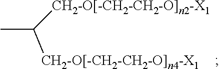
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10; and
n4 is an integer independently selected in each instance from the group of 1, 2, 3, 4, 5, 6, 7, 8, 9, and 10;
R₄ and R₅ are each independently selected from C₁-C₆ alkyl, with the proviso that, when R₄ is ethyl, R₅ is not ethyl;
or R₄ and R₅, together with the nitrogen atom to which they are bound, form a ring selected from the group of: 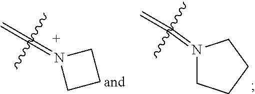
R₆ is hydrogen;
with the proviso that the sum of n2+n4 is not greater than 10; and
with the proviso that, when R₁ is methyl and R₄ is ethyl, R₅ is not ethyl.

10. The gel-based formulation for tissue imaging of claim 9, wherein R₁ is selected from the group of straight or branched C₁-C₃ alkyl, R₆ is hydrogen and R₄ and R₅ are each independently selected from the group of straight or branched C₁-C₃ alkyl.

11. The gel-based formulation for tissue imaging of claim 1, wherein the compound is a compound of Formula (V): 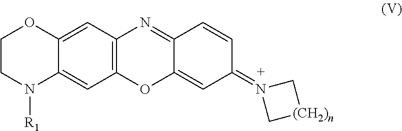
wherein:
R₁ is selected from the group of straight or branched C₁-C₆ alkyl;
—(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 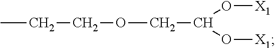
b) 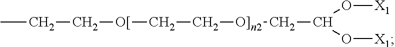
c) 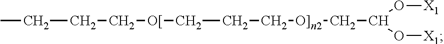
and
d) 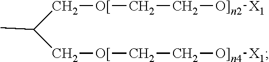
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n is an integer selected from the group of 1 and 2;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10; and
n4 is an integer independently selected in each instance from the group of 1, 2, 3, 4, 5, 6, 7, 8, 9, and 10;
with the proviso that the sum of n2+n4 is not greater than 10.

12. The gel-based formulation for tissue imaging of claim 1, wherein the compound is a compound of the formula: 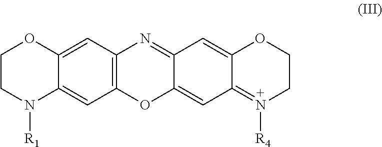
wherein:
R₁ and R₂ are each independently selected from the group of straight or branched C₁-C₆ alkyl; —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 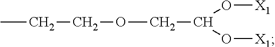
b) 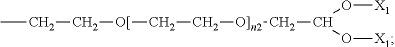
c) 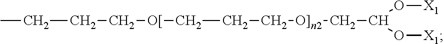
and
d) 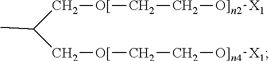
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n is an integer selected from the group of 1 and 2;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10; and
n4 is an integer independently selected in each instance from the group of 1, 2, 3, 4, 5, 6, 7, 8, 9, and 10;
with the proviso that the sum of n2+n4 is not greater than 10;
with the proviso that, when R₁ is methyl, R₄ is not methyl; and
with the proviso that, when R₁ is ethyl, R₄ is not ethyl.

13. The gel-based formulation for tissue imaging of claim 12, wherein R₄ is selected from the group of straight or branched C₁-C₃ alkyl; with the proviso that, when R₁ is methyl, R₄ is not methyl.

14. The gel-based formulation for tissue imaging of claim 1, wherein the compound is a compound of Formula (IX): 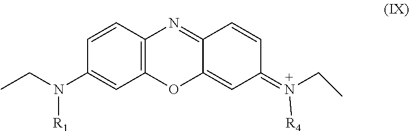
wherein R₁ and R₄ are each independently selected from C₁-C₆ alkyl.

15. The gel-based formulation for tissue imaging of claim 14, wherein R₁ and R₄ are each independently selected from C₁-C₃ alkyl.

16. The gel-based formulation for tissue imaging of claim 1, wherein the compound is a compound of Formula (IX): 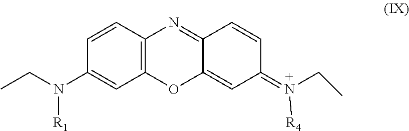
wherein R₄ is C₁-C₆ alkyl;
R₁ is selected from the group of straight or branched C₁-C₆ alkyl;
—(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; or a moiety selected from the group of:
a) 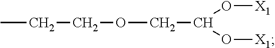
b) 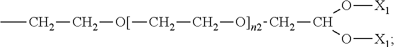
c) 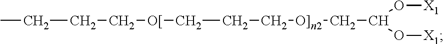
and
d) 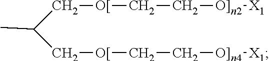
X₁ in each instance is independently selected from C₁-C₆ straight or branched alkyl, C₁-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10; and
n4 is an integer independently selected in each instance from the group of 1, 2, 3, 4, 5, 6, 7, 8, 9, and 10;
with the proviso that the sum of n2+n4 is not greater than 10.

17. The gel-based formulation of claim 1, wherein the compound of Formula (I) is selected from the group of:

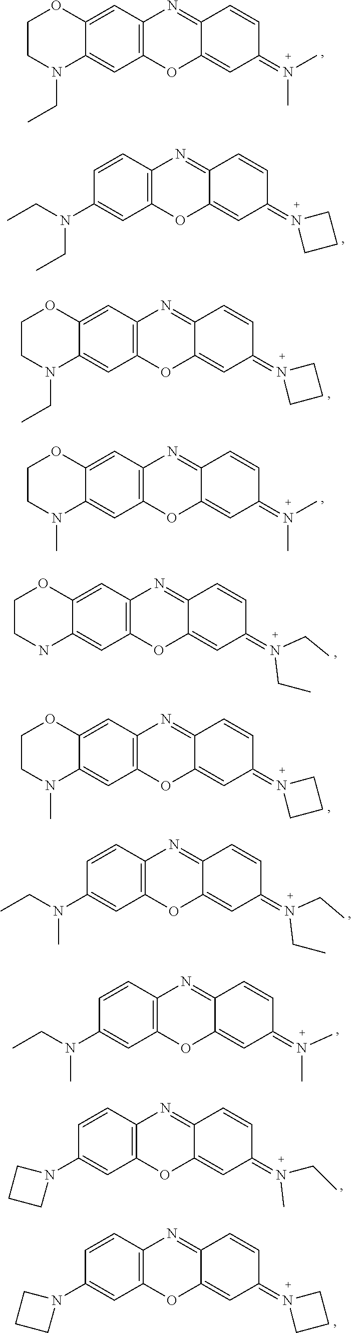

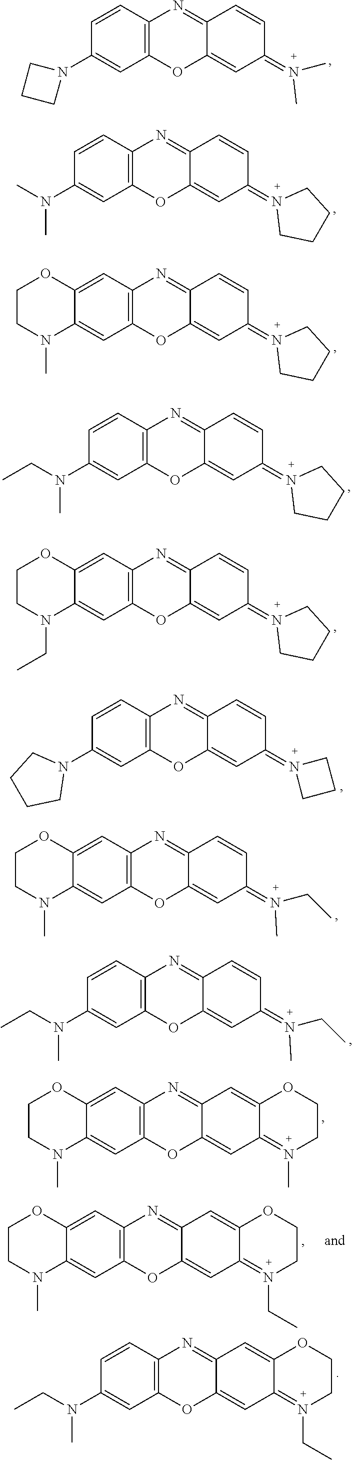

18. A method of detecting nerves intraoperatively in a subject undergoing surgery comprising:
directly applying a gel-based formulation comprising (i) an effective amount of a fluorophore compound as described in claim 1, and (ii) 5-10% sodium alginate and/or 18-26% PEO-PPO-PEO triblock copolymer to stain tissue undergoing surgery; and
imaging the stained tissue, thereby detecting nerves intraoperatively in the subject undergoing surgery.
